(19) United States
(12) Patent Application Publication
(10) Pub. No.: US 2024/0099652 A1
(43) Pub. Date: Mar. 28, 2024

(54) APPARATUS FOR AUTOMATED PAIN TESTING IN RODENTS

(71) Applicant: THE HOSPITAL FOR SICK CHILDREN, Toronto (CA)

(72) Inventors: CHRISTOPHER DEDEK, OTTAWA (CA); MEHDI ABBASI AZADGOLEH, TORONTO (CA); STEVE ALEC PRESCOTT, TORONTO (CA)

(21) Appl. No.: 18/371,847

(22) Filed: Sep. 22, 2023

Related U.S. Application Data
(60) Provisional application No. 63/409,005, filed on Sep. 22, 2022.

(51) Int. Cl. A61B 5/00; A61F 7/00; A61N 1/36

(52) U.S. Cl. CPC A61B 5/4824; A61B 5/0077; A61F 7/007; A61N 1/36014; A61B 2503/40; A61B 2560/04; G16H 10/20

(57) ABSTRACT

Disclosed herein is a device that standardizes and automates pain testing in laboratory rodents by providing computer-controlled aiming and delivery of various somatosensory stimuli, and precisely measuring the evoked responses. For photostimuli, red light is incorporated into the light path and its reflectance off the paw is measured (at ≥1 kHz) by a photodetector mounted on the device. The reflectance signal changes rapidly when the target paw moves, thus enabling automated measurement of withdrawal latencies with millisecond precision. A camera mounted on the device, below the mouse, provides video for aiming and for behavior analysis before, during and after stimulation. The device can be aimed manually or automatically using artificial intelligence (AI), in the latter case, the target paw is identified and tracked by a pre-trained neural network, and real-time information about paw location is used to command motorized linear actuators to move the stimulator and to initiate stimulation.

### FIELD

The present disclosure relates to an apparatus for automated pain testing in laboratory rodents.

### BACKGROUND

Measuring withdrawal from noxious stimuli in laboratory rodents is a mainstay of preclinical pain research [1-4]. Testing is often conducted on the hind paw, in part because many chronic pain models are designed to increase paw sensitivity through manipulations of the paw or the nerves innervating it [4-7]. Measuring evoked pain with withdrawal reflexes has been criticized [8] since ongoing (non-evoked) pain is a bigger clinical problem [9], but tactile and thermal sensitivity are altered in many chronic pain conditions [10], plus allodynia and spontaneous pain tend to be

correlated in human studies [11, 12] and in some [13] but not all [14] mouse studies. Furthermore, sensory profiling is useful for stratifying patients in clinical trials [15, 16] and altered sensitivity is central to diagnosing certain conditions, e.g. fibromyalgia [17]. It logically follows that ongoing pain should be assessed in addition to, not instead of, evoked pain [18]. Doing so would provide a more complete picture, including the relationship between evoked and ongoing pain. However, a problematic aspect of this testing must be rectified: most stimuli are applied by hand and responses are measured by eye, leading to variability between studies because of subjectivity and inconsistencies across experimentalists. For instance, outcomes of the hot water tail flick test were shown to depend more on who conducts the testing than on any other factor [19]. This likely generalizes to other behavioral tests but has received scant attention compared with other factors, like sex [20]. Outdated technology and poorly standardized testing protocols contribute to the oft-cited reproducibility crisis [21] and are long overdue for transformative improvements.

Preclinical pain tests typically measure withdrawal threshold using brief repeated (incrementing) stimuli like von Frey filaments [22] or sustained stimuli like radiant heat [23]. The stimulus intensity (force or skin temperature) at which withdrawal occurs is assumed to be the lowest intensity perceived as painful (i.e., pain threshold) [24]. Of course, withdrawal might not always be triggered by pain, and focusing on threshold fails to consider variations in pain intensity over a broad stimulus range. Recent studies have quantified responses to suprathreshold mechanical stimulation using high-speed video [25-27] to analyse details of the withdrawal movement, but despite precise response measurement, stimuli were delivered by hand and throughput was low. Resolving subtle changes in pain sensitivity requires that stimulus-response relationships be measured with high resolution (which requires both reproducible stimulation and precise response measurement), over a broad dynamic range, and with reasonable efficiency (throughput). Improvements in one factor may come at the expense of other factors. The best compromise depends on the particular experiment, but improving reproducibility and throughput would be a huge benefit.

Optogenetics has provided an unprecedented opportunity to study somatosensory coding, including nociception. Expressing actuators like channelrhodopsin-2 (ChR2) in genetically defined subsets of afferents allows those afferents to be selectively activated or inhibited with light applied through the skin (transcutaneously) or directly to the nerve or spinal cord using more invasive methods [28, 29]. Afferents can be optogenetically activated in combinations not possible with somatosensory stimulation; for instance, mechanical stimuli that activate Aδ high-threshold mechanoreceptors (HTMRs) normally also activate low-threshold mechanoreceptors (LTMRs), so it is only by expressing ChR2 selectively in HTMRs that HTMRs can be activated in isolation [30]. Causal relationships between afferent co-activation patterns and perception/behavior [31] can be thoroughly tested in this way. Elucidating those relationships is key to understanding physiological pain and how pathology disrupts normal coding, facilitating development of targeted therapies. Optogenetics has been used for basic pain research but, despite its potential, has not yet been adopted for drug testing [32]. Transcutaneous photostimulation is amenable to high-throughput testing but, like tactile and thermal stimuli, is hard to apply reproducibly in behaving animals.

Thus, it would be very advantageous to provide an apparatus that improves reproducibility of stimulation, including transcutaneous photostimulation and mechanostimulation, while also streamlining response measurement in order to increase throughput. We developed a device that combines delivery of consistent optogenetic, thermal, and mechanical (tactile) stimuli with measurement of withdrawal latency with millisecond precision. The device can deliver precise light stimuli as pulses, ramps, or other selected waveforms, which we show can reveal differences in responses not seen before. Importantly, the novel design of our device offers a clear view of the mouse from below, which allowed us to automate aiming of the stimulator; specifically, using artificial intelligence (AI), we trained a neural network to recognize the paw and used paw location thus ascertained to aim the stimulator using motorized actuators. Automated aiming, when combined with automation of stimulus delivery and response measurement, enables fully automated testing. Finally, the substage video also provides a wealth of data about non-reflexive behaviors for consideration alongside withdrawal measurements to more thoroughly assess the rodent pain experience.

### SUMMARY

The present disclosure provides a device that is capable of reproducible and fully automated pain and other somatosensory testing in laboratory rodents, especially mice. Mice are kept individually in enclosures on a platform. The moveable stimulator is positioned underneath to stimulate one of their paws from below. The photostimulator uses an LED for optogenetic stimulation or inhibition and an infrared (IR, 980 nm) laser for thermal stimulation. A video camera is mounted to the stimulator and used for aiming. Red (625 nm) light delivered through a common light path is used to confirm aiming before initiation of photostimulation with blue or IR light, and is maintained during and after stimulation for the purposes of withdrawal detection. Reflectance of the red light, which decreases upon paw withdrawal, is measured by a photodetector to assess withdrawal latencies with millisecond precision. The accuracy of latency measurements based on red reflectance was verified by comparison with high-speed (1000 frames/sec; fps) video. Photostimuli delivered by the new device were significantly more reproducible (less variable) than those delivered by a handheld fiber optic based on comparison across multiple experimenters. Stable aiming is also key for delivering slow stimuli (e.g., ramps) with the proper intensity. Other stimulating tools can be installed on the moveable stimulator, and be controlled by computer to ensure precise stimulation. For example, the current prototype includes a mechanostimulating probe whose position is controlled by computer while measuring the force exerted on the paw. Withdrawal to mechanostimulation is evident as a drop in exerted force (measured at 1 KHz) thus avoiding the need for the reflectance signal when using this stimulation mode. All stimulation and response measurements are computer-controlled. We have also automated the aiming process by training a neural network to identify and track the paws, and then centering the target paw in the stimulation zone by repositioning the stimulator using motorized linear actuators; the stimulation sequence is initiated once the mouse is deemed sufficiently stationary by the neural network. The entire process including aiming, stimulation, and response measurement has been automated in the apparatus disclosed herein. The stimulator can be shifted to sequentially stimulate different mice for high-throughput testing, which entails interleaving stimuli so that different mice are stimulated in rapid succession but each mouse experiences repeat testing at a long interval. Moreover, substage video reveals non-reflexive behaviors for consideration alongside measurement of reflexive withdrawal responses to better assess the pain experience.

Thus, the present disclosure provides an apparatus for automated measurement of pain and responses to various somatosensory stimuli in laboratory rodents, comprising:

one or more enclosures for individual rodents;

a platform on which said one or more enclosures are positioned;

a moveable device positioned underneath said platform and enclosures and configured to:
aim at a target paw of the rodent,
deliver one or more different stimulus modalities, alone or in combination, to the target paw,
detect changes in position of the target paw with millisecond precision, and
collect video of rodent activity before, during and after stimulation; and

a controller operably connected to the moveable device and configured to:
coordinate all aspects of stimulation using programmed instructions,
synchronize recorded data with stimulus timing and calculate withdrawal latency therefrom, and
automatically record all data, metadata, and calculations to electronic files.

The present disclosure also provides a method for automated measurement of pain and responses to various somatosensory stimuli in laboratory rodents, comprising:

confining one or more rodents individually in one or more enclosures in which said one or more enclosures are located on a platform;

directing a moveable device positioned underneath said platform and enclosures to:
aim different sources of stimulation, alone or in combination, at a target paw of the rodent
deliver one or more different stimulus modalities, alone or in combination, to the target paw,
detect changes in position of the target paw with millisecond precision, and
collect video of rodent activity before, during and after stimulation; and using a controller operably connected to the moveable device to:
   coordinate all aspects of stimulation using programmed instructions,
   synchronize recorded data with stimulus timing and calculate withdrawal latency therefrom, and
   automatically record all data, metadata, and calculations to electronic files.

The enclosures may each comprise a separate clear tube and opaque cubicle, wherein the clear tube is used to transfer each rodent from its home cage to the testing platform and to house the rodent during testing on the platform, and wherein the opaque, magnetically connectable cubicles separate the rodents and position them at a desired spacing and alignment on the platform.

The platform may be made of an optically clear material, and wherein the moveable device may include a light source of selected wavelength(s) to provide optogenetic stimulation.

The platform may be made of an optically clear material, and wherein the moveable device may include infrared (IR) light for thermal stimulation via radiant heating.

The platform may be a metal grating, and the moveable device may include a mechanical indenter which stimulates by physical contact with the target paw.

The mechanical indenter may be configured to measure force applied to the paw and to detect withdrawal based on changes in force as the target paw is withdrawn from the indenter arm.

The mechanical indenter may be adapted to provide other somatosensory modalities requiring contact with the paw, including:
heating or cooling using a Peltier device,
application of chemicals like acetone for cooling or capsaicin for heating,
needle prick using a sharp-tipped probe, and
dynamic touch using a rotary brush.

The one or more stimulus modalities include combinations of light, heat, mechanical and chemical agents.

The moveable device may be configured to provide different stimulus modalities sequentially to test different stimulus modalities on separate trials.

The moveable device may be configured to provide two or more different stimulus modalities together on a given trial.

The moveable device may include a source of red light configured to be aimed at the target paw in order to assist aiming by identifying a photostimulation zone prior to initiating photostimulation with other wavelengths of light.

The moveable device may be mounted on a set of motorized actuators and is aimed at the target paw by a human operator via computer using a joystick or keypad.

The moveable device may be mounted on a set of motorized actuators and is aimed at the target paw automatically by a neural network pre-trained to recognize and track the target paw.

The initiation of stimulation may be made contingent on various factors ascertained from video and assessed by artificial intelligence, such as whether the rodent is stationary, has assumed a certain posture, and/or is engaged in a certain behavior.

Software coordinates interleaved testing of a cohort of rodents positioned on the platform so that many rodents can be rapidly tested sequentially, but where each rodent is not re-tested before a minimum acceptable period has elapsed, thus enabling high-throughput testing of the cohort.

A red light source may be used to illuminate the target paw and a photodetector is used to measure changes in the reflectance of red light off the target paw before, during and after stimulation in order to detect withdrawal of the target paw with millisecond precision.

A further understanding of the functional and advantageous aspects of the disclosure can be realized by reference to the following detailed description and drawings.

### DESCRIPTION OF THE DRAWINGS

Embodiments will now be described, by way of example only, with reference to the drawings, in which:

FIGS. 1A and 1B show an overview of algometry system in which:

FIG. 1A shows how the components of the system are configured. The test subjects (mice or other rodents) are kept separately in enclosures on a platform above the stimulator. The stimulator remains at a fixed distance below the platform but is free to move horizontally, left-right and forward-back. In the manually aimed version of the system depicted here, the experimenter slides the stimulator relative to the platform and mice positioned thereon, using a live video feed to aim; a motorized version of the device is described later, in FIG. 11. In the version of the system used for photostimulation (i.e., optogenetic and radiant heat stimulation) depicted here, the platform is made of plexiglass; a wire grate platform is used when testing mechanical stimuli or other stimuli requiring direct physical contact with the paw, as described later in FIG. 10. A hybrid platform of plexiglass and wire grate is envisioned so that mice can be tested with photostimuli or with mechanostimuli depending where on the platform (i.e., plexiglass vs wire gate) they are positioned.

FIG. 1B shows a close-up view of a novel enclosure design. Mice are transferred from their home cage to the platform in a clear plexiglass tube. The tube is rotated vertically when placed on the platform, and is slid back into an opaque cubicle that covers all sides except the front and bottom. Cubicles are designed with internal magnets so that they can be easily added or removed depending on the number of mice tested. Magnets ensure that attached cubicles are properly aligned and equally spaced, which is critical when automating the testing process, as explained later. Each tube has a small notch cut into its base to allow the experimenter some access to the mouse (e.g., to touch it to wake or help orient it). If the notch is unwanted, the tube can be positioned (i.e., flipped) so that the notch is at the top.

FIG. 1C shows, in the top panels, examples of aiming from the perspective of the camera. The video camera is mounted on the stimulator with the center of its field of view aligned with the photostimulation zone. The photostimulation zone is shown as a dotted circle. Prior to photostimulation with blue or IR light, red light is turned on in order for the experimenter to see the photostimulation zone. Left top panel labeled “a” shows the red light hitting the leg, indicating that the stimulator is off-target. Middle top panel labeled “b” shows red light centered on the paw, indicating that the stimulator is now properly aimed and photostimulation can proceed. Right top panel labeled “c” shows the mouse after withdrawing from optogenetic stimulation. Bottom panel shows the intensity of reflected red light plotted against time. Timing of examples in top panels are indicated with labeled arrows on the plot. Timing of stimulation is shown with shading labeled “stim”. The increase in the reflectance signal when the paw is properly targeted (compare panels “a” and “b”), the stability of the reflectance signal during the pre-stimulus period (around the time of panel “b”), and the rapid drop in reflectance signal upon stimulation (compare panels “b” and “c”) are used to assist aiming, stimulus timing, and response measurement, respectively.

FIGS. 2A and 2B show the current photostimulator in which:

FIG. 2A shows the phototstimulator, which includes three light sources: red (625 nm) for aiming and withdrawal measurement, blue (455 nm) for optogenetic stimulation, and infrared (980 nm) for radiant heating. Other wavelengths can be incorporated as required for specific experiments. The collimated light beams are combined using dichroic mirrors and are then redirected upward and focused onto the plexiglass platform. Light intensities and timing are controlled by computer. A photodetector measures the amount of red light reflected off the target paw. Numbers indicated on the device identify dichroic mirrors and filters explained in FIG. 2B. A camera provides video for aiming. An additional IR light source providing diffuse, unfocussed light can be used to improve lighting during high-speed video but is not depicted here.

FIG. 2B shows how different wavelengths of light are combined. Graphs shows emission spectra for the various light sources plotted as normalized light intensity on the y-axis on the left against the wavelength of light on the x-axis. The 455 nm LED (blue) is shown as a thick dotted line. The 625 nm LED (red) is shown as a thick solid line. The 980 nm laser (IR) is shown as a thick dashed line. The transmission of dichroic mirrors and filters numbered “1-3” in FIG. 2A are also shown as gray lines plotted relative to the y-axis on the right against the wavelength on the x-axis. Left panel shows that dichroic mirror labeled “1” reflects blue light and transmits red light, thus combining the blue and red light. Middle panels shows that dichroic mirror labeled “2” reflects blue and red light and transmits IR light, thus combining the blue/red and IR light. Right panel shows a notch filter labeled “3” placed in front of the photodetector blocks blue and IR light, selectively transmitting red light so that the detector can measure the intensity of red light reflected off the target.

FIGS. 3A to 3C show that reproducible photostimulation requires precise aim, in which:

FIG. 3A shows a heatmap showing how the amount of light hitting a paw-shaped target depends on the precise x-y-z positioning of a fiber optic typically used for optogenetic stimulation. To measure light-on-target, a paw-shaped cut out is placed over a photodiode positioned face down on the plexiglass platform. A fiber optic was mounted on linear actuators so that light delivery could be re-measured at different points on an x-y grid, where the x-axis is aligned with the long axis (i.e., length) of the paw-shaped cut out, and the y-axis is aligned with the short axis (i.e., width) of the paw-shaped cut out. The z-axis corresponds to the distance of the fiber optic tip below the platform; testing was conducted at two different z distances: top panel shows results for 1 mm distance and bottom panel shows results for 5 mm distance. Gray shading shows the amount of light reaching the photodiode through the paw-shaped cut out, measured as power. Results show that the amount of light hitting the target is sensitive to aiming and, therefore, that any variations in aiming will translate to unintended variations in stimulus intensity. Moreover, comparison of the top and bottom panels shows that photostimulation with a fiber optic is only effective when the tip is positioned very close to the target; specifically, light delivery is significantly less in the bottom panel (5 mm distance) than in the top panel (1 mm distance).

FIG. 3B shows the amount of light reaching the target during sustained (10 second long) photostimulation. Light delivery was measured as described above for FIG. 3A. Traces show light-on-target quantified as power (vertical) plotted against time (horizontal). Each differently shaded gray trace is from a different person trying to aim the fiber optic as carefully as possible at the target for 10 seconds; data were collected from 13 individuals (i.e. testers). The black trace shows the performance of our photostimulator for comparison. Graph on right shows stability of light delivery quantified as the signal-to-noise ratio (SNR=mean²/SD²). The SNR when using the stimulator (55.6 dB, shown as black line) was significantly higher than for the handheld fiber optic (23.5±2.0 dB, mean±SEM, each dot corresponds to a different tester). t₁₂=15.8, p<0.001, one sample t-test.

FIG. 3C shows trial-to-trial variability of light delivery during brief (100 ms-long) photostimulation pulses. Light delivery was measured as described above for FIG. 3A. Five testers delivered 10 pulses by handheld fiber optic and another 10 pulses using the photostimulator. The light source was re-aimed between each pulse, which were triggered one at a time. Graph on left shows light-on-target (quantified as stimulus power) for different testers and stimulation methods. Gray symbols show results when using the handheld fiber optic; black symbols show results when using the photostimulator. The spread of points within each tester and across testers shows the intra- and inter-tester variability, respectively. Specifically, open symbols show results for each trial from each tester; corresponding bars summarize the intra-tester average). Filled symbols (“group” data) show intra-tester averages; corresponding bars summarize group (i.e., cross-tester) average. Gray dotted line and shading show average light intensity±SD across 10 trials without moving the photostimulator. Graph on right quantifies variability by plotting deviation from the mean for handheld fiber optic (shown in gray) and photostimulator (shown in black). Average trial-to-trial deviation from each tester's average was significantly larger for handheld fiber optic (26.3±3.0 mW; mean±SEM) than for the photostimulator (6.4±0.7 mW) (t₉₈=6.42, p<0.001, unpaired t-test), which shows that using the photostimulator reduces intra-tester variability. Tester-to-tester deviation from the group average was larger for handheld fiber optic (44.5±18.2 mW) than for the photostimulator (9.7±2.0 mW) (t₈=1.94, p=0.093), which shows that using the photostimulator also reduces inter-tester variability. Data here are for the manually aimed photostimulator; comparison with automated aiming of the same photostimulator is reported in FIG. 11C.

FIG. 4 shows how photothreshold is measured. Specifically, the probability of paw withdrawal on the y-axis plotted against stimulus power on the x-axis, thus creating an input-output (or stimulus-response) curve. For this testing, five 100 ms-long blue pulses were delivered at each of 5 intensities to a single mouse. Threshold is taken as the intensity at 50% probability of withdrawal, as inferred from fitted curve. These results show that a photostimulus “threshold” intensity can be identified by titrating the stimulus intensity while monitoring whether or not a withdrawal response occurs. Precise threshold measurement requires that stimuli do not have noisy (unintended and unaccounted for) variations in their intensity as any such variability decreases the precision of threshold measurements which, in turn, decreases one's ability to resolve subtle changes in threshold (e.g., to measure drug effects).

FIGS. 5A to 5C show that the latency of paw withdrawal following photostimulation can be accurately and precisely measured by changes in the reflectance of red light, in which:

FIG. 5A shows sample frames extracted from high-speed video recorded simultaneously with red reflectance before, during, and after photostimulation with a 100 ms pulse of blue light. Timing of video frames “a-c” are indicated with labeled arrows in bottom panel. The top left panel labeled “a” shows the mouse prior to stimulation, with its left hind paw resting on the platform. The top middle panel labeled “b” shows the mouse shortly after stimulus initiation, with its left hind paw lifted off the platform. The top right panel labeled c shows the mouse after stimulation, with its left hind paw once again placed on the platform. A neural network was trained to identify the paw and track its position, as indicated by the dot. In the bottom panel, the dashed trace shows the height of the dot plotted against time. The light gray trace shows the simultaneously recorded red reflectance signal plotted against time; recall from FIG. 1C that the reflectance of red light off the paw drops when the paw is withdrawn from the photostimulation zone. The video and reflectance signal were both collected at 1 KHz. Comparison of the reflectance signal and paw height confirms that reflectance drops at the same time as the paw is raised. Timing of the 100 ms-long blue photostimulus pulse is indicated with gray shading. For each trial included in this analysis, withdrawal latency is measured in two ways: (1) based on when paw height exceeds a threshold defined relative baseline or (2) when the reflectance signal drops below a threshold defined relative to baseline. Horizontal solid black lines show baselines. Horizontal dotted lines show thresholds. Vertical dashed lines show time of threshold crossing, from which latency is calculated based on time of threshold crossing minus time of stimulus onset, as indicated by double headed arrows. For the reflectance signal, threshold crossing and latency measurement are conducted automatically and in real-time by the software.

FIG. 5B shows, in the left panel, withdrawal latencies determined by method 1 (paw height) plotted against latencies determined by method 2 (reflectance), where each dot represents a separate trial. If both methods yielded identical latency measurements, all data points would fall along the black dashed line. X- and y-axes are shown on a log scale to help better visualize the data. Data were collected from 7 mice responding to photostimulus pulses with a range of intensities; from a total of 218 trials, 7 trials were excluded (3.2%) based on errors identified by visual inspection of raw data. The error trials are shown as filled gray dots. Top right panel shows an example error trial (plotted like in FIG. 5A) in which automated determination of paw position from high-speed video was corrupted by the blue light during photostimulation; such an error occurred in 3 trials. Bottom right panel shows an example error trial in which the reflectance signal did not immediately change upon paw withdrawal; such an error occurred in 4 trials. The false negative rate for the reflectance signal is thus <2% and we did not identify any false positives (i.e., changes in reflectance in the absence of paw movement). After removing error trials, linear regression on the remaining 211 trials yields an excellent fit (R=0.966) shown as a solid gray line. The fitted line has slope of 1.007, which is very near the expected slope of 1.

FIG. 5C shows the same data as in FIG. 5B but now graphed as a Bland-Altman plot. This plot shows the difference in latency between methods 2 and 1 (i.e., reflectance−paw height) plotted against the average across methods calculated for each trial. The average difference of −0.4 ms does not deviate significantly from 0 (t₂₁₀=−0.8992, p=0.374, one sample t-test), meaning there is no fixed bias in the latency measurement methods. Black line shows linear regression; gray shading shows 95% prediction band. The slope of the fitted line (0.021) deviates significantly from horizontal (t₂₀₉=2.31, p=0.021), suggesting a proportional bias, but the errors are inconsequential as highlighted in gray: For a short-latency response of 25 ms, latency by reflectance is on average only 0.9 ms shorter than latency by paw height (a mere 3.6% error) whereas for a long-latency response of 150 ms, latency by reflectance is on average only 1.7 ms longer (a mere 1.1% error). The histogram above the main graph shows the probability of responses with different withdrawal latencies, and reveals a bimodal distribution; specifically, responses tend to occur with either short latencies (between 20 and 40 ms) or long latencies (between 100 and 200 ms) but not with intermediate latencies (between 40 and 100 ms); hence, sample measurement errors were calculated for a typical short- and long-latency response. Overall, these results show that the reflectance signal enables latency measurements as precise as those using high-speed video. All subsequently reported latencies are based on automated reflectance-based measurements.

FIG. 6 shows the benefits of combining precise stimulus delivery and precise response measurement. The main graph shows response latency plotted against the power of 100 ms-long photostimulus pulses. Each symbol represents the response on one trial; data were collected from 10 mice, including 5 that express channelrhodopsin-2 (ChR2) in all primary afferents (namely Advillin-ChR2, shown with x′s) and 5 that express ChR2 more selectively in TRPV1-lineage afferents (namely TRPV1-ChR2, shown with o's). Threshold was determined by testing with “perithreshold” stimulus powers; thereafter, each mouse was tested with four stimulus intensities defined by fixed increments above threshold; each mouse was tested three times at each suprathreshold intensity, except for the highest intensity, which was tested only once. The graph to the right of the main graph shows the probability (i.e.

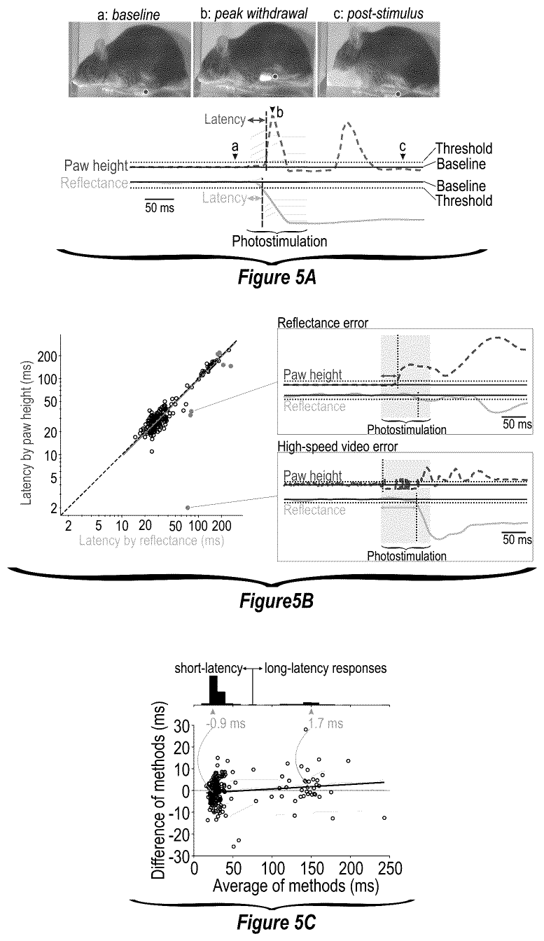

histogram) of responses with different latencies, which, like in FIG. 5C, shows a bimodal distribution. Responses were subdivided into long- and short-latency responses based on latencies > or < than 75 ms, respectively; the 75 ms cutoff is shown as a dotted line on main graph that extends to the histogram. The stacked bar graph above the main graph shows the proportion of long-latency responses (shown in gray) and short-latency responses (shown in black) as a function of stimulus power.

Proportions varied significantly with stimulus power (χ²=105.01, p<0.0001); in other words, long-latency responses predominated with weak stimulation whereas short-latency responses predominated with strong stimulation. Lines on main graph show separate regressions for long-latency responses (gray line, R=0.69; y=254.8 x^{-0.29}) and short-latency responses (black line, R=0.54; y=49.8 x^{-0.17}); lines were fitted to log-transformed data but are reported here without the transformation to emphasize the exponential reduction in latency as stimulus power is increased. These results show that, in addition to responses switching from slow (long-latency) to fast (short-latency) as stimulus power increases, slow and fast responses themselves speed up with increasing stimulus power. Precise stimulation and response measurement are required to ascertain and properly quantify such relationships.

FIG. 7A and 7B show the benefit of conducting optogenetic testing using different photostimulus waveforms, in which:

FIG. 7A shows the latency of responses to 15 second-long photostimulus ramps. Each symbol represents the response on one trial; data were collected from 9 mice, including 4 that express ChR2 in all primary afferents (namely Advillin-ChR2, shown in gray) and 5 that express ChR2 selectively in nociceptive afferents (namely Nav1.8-ChR2, shown in black). Gray shading shows timing of ramp. Left graph shows the cumulative probability of withdrawal latencies for Advillin-ChR2 mice (in gray) and Nav1.8-ChR2 mice (in black). Withdrawal latencies in Advillin-ChR2 mice were significantly more variable than in Nav1.8-ChR2 (D-0.714, p=3.67×10⁻⁸, two-sample Kolmogorov-Smirnov test). Right graph shows that variability occurs within each mouse; specifically, the intra-mouse coefficient of variation (=SD/mean) was significantly higher in Advillin-ChR2 mice (t₇=−6.575, p<0.001, two-sample t-test).

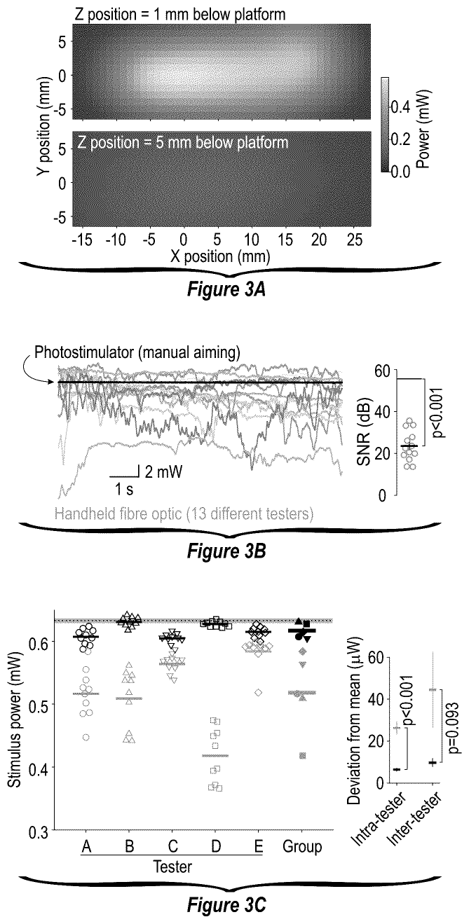

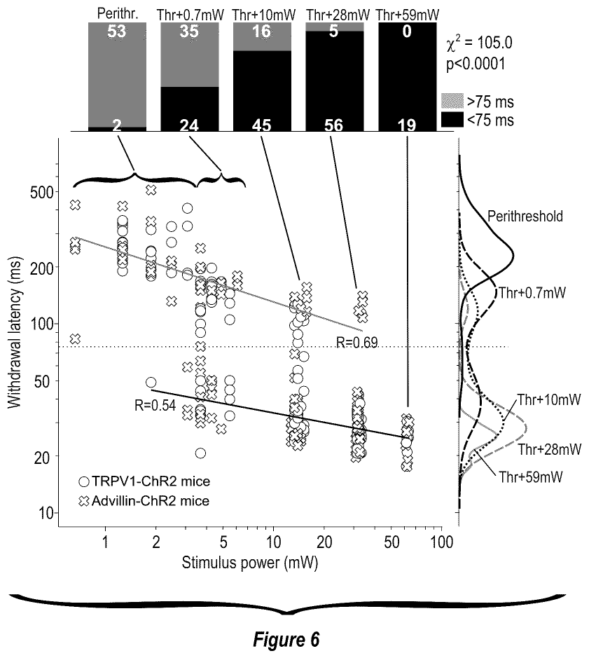

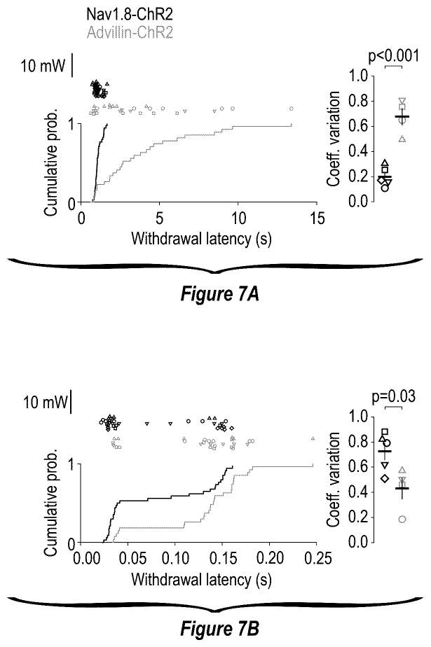

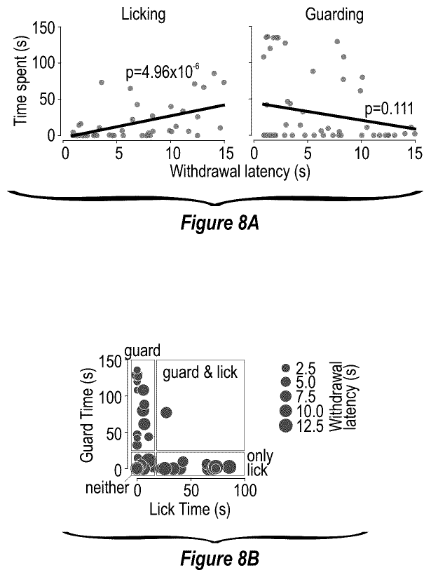

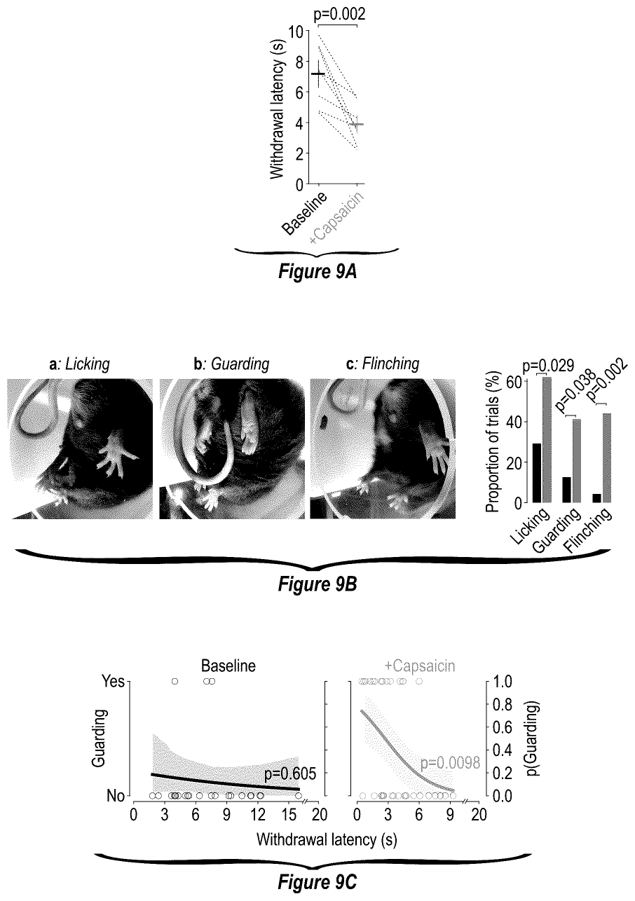

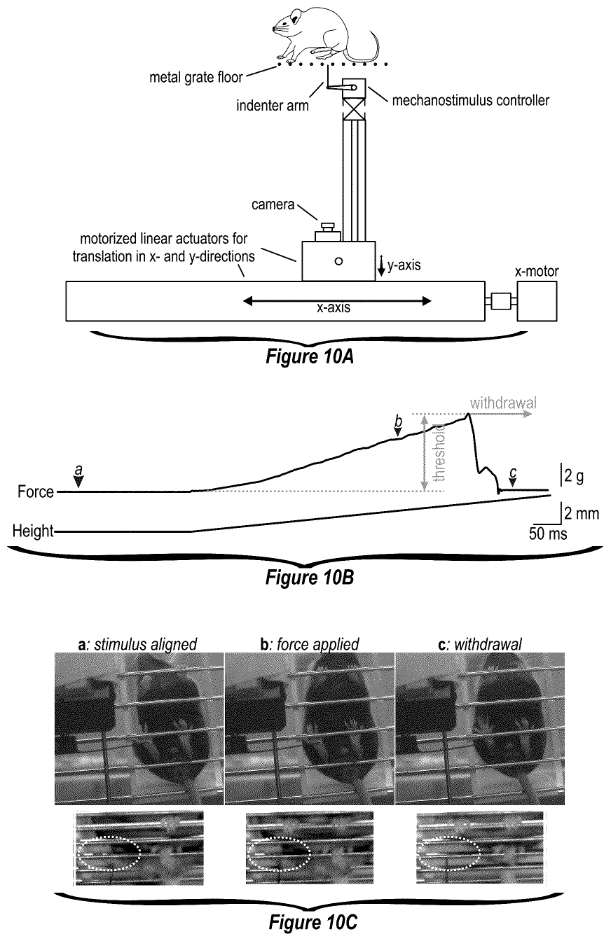

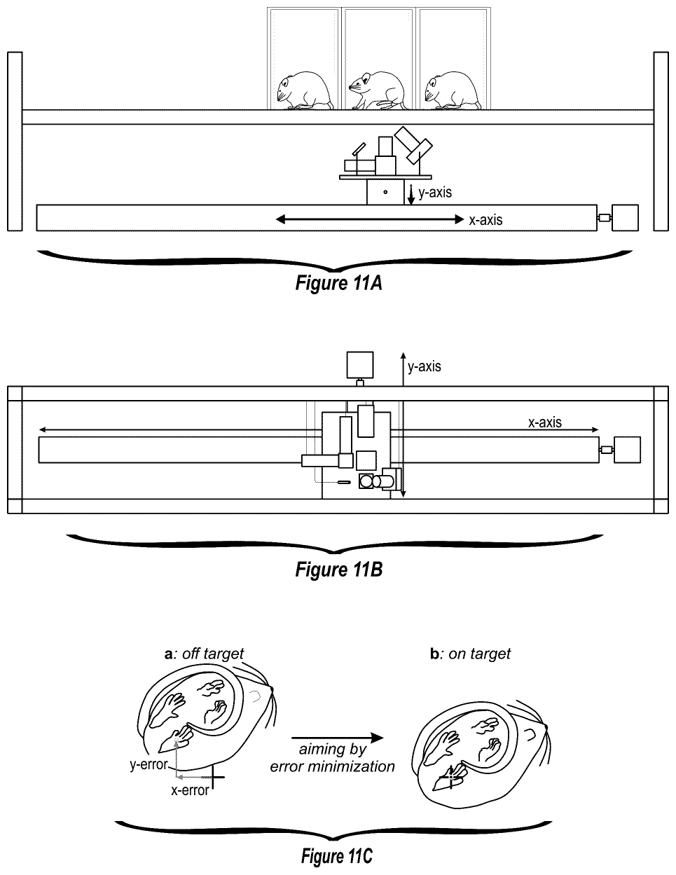

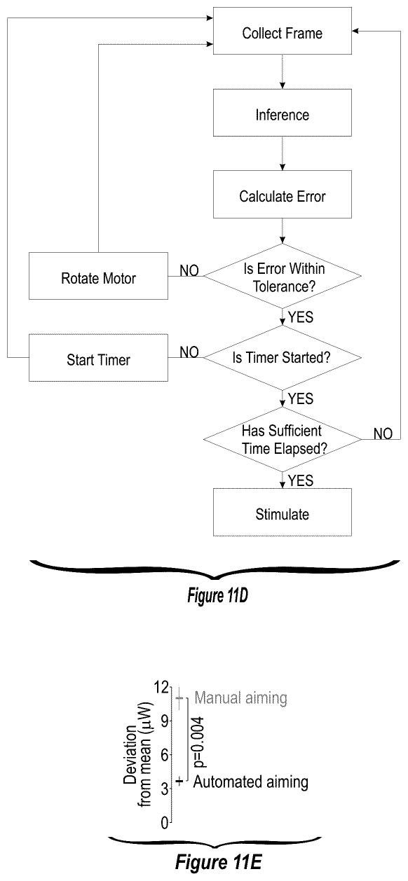

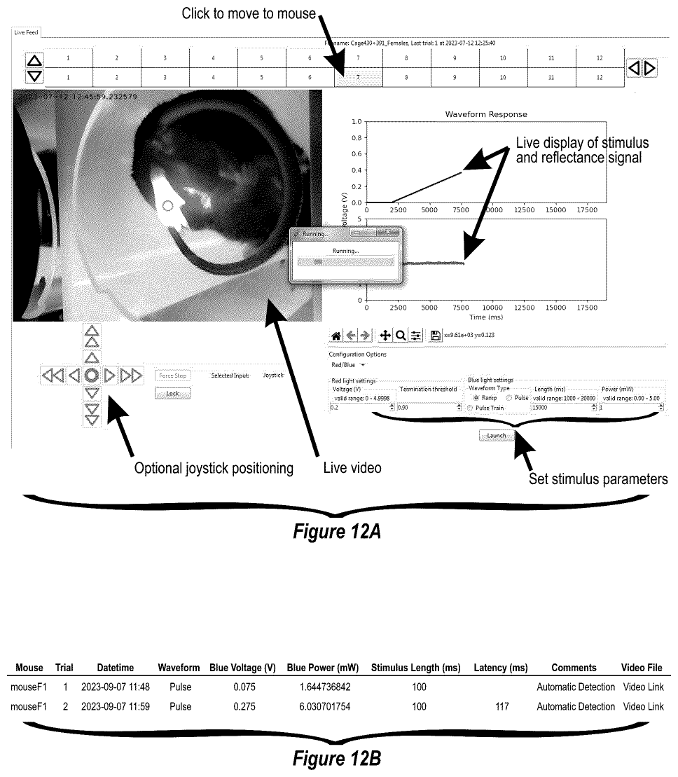

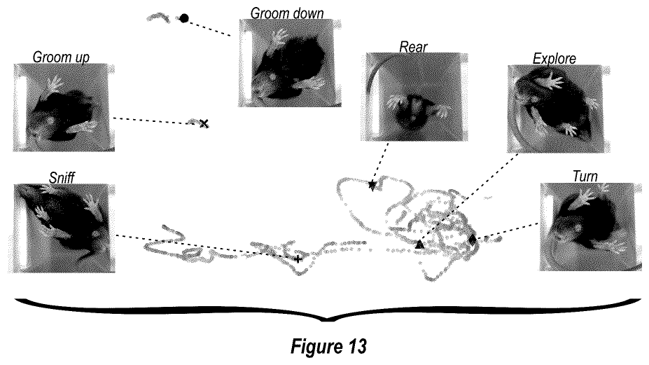

FIG. 7B shows the latency of responses to 100 ms-long pulses in the same mice tested with ramps in FIG. 7A. Gray shading shows timing of pulse. Left graph shows the cumulative probability of withdrawal latencies for Advillin-ChR2 mice (in gray) and Nav1.8-ChR2 mice (in black). Both genotypes exhibited bimodal withdrawal latencies (like in FIG. 6). Distributions differed significantly between genotypes (D=0.403, p=0.009, two-sample Kolmogorov-Smirnov test) but this is due to the different ratio of short- and long-latency responses rather than a fundamentally different distribution. Right graph shows that the intra-mouse coefficient of variation was high in both genotypes, but slightly higher in Nav1.8-ChR2 mice (t₇=2.71, p=0.030), which is the opposite to results in FIG. 7A. Note that latencies are roughly 3 orders of magnitude shorter in FIG. 7B (for pulses) than in FIG. 7A (for ramps). These results highlight that different stimulus waveforms (e.g., ramp vs pulse) can reveal different information and, therefore, that testing (e.g., to compare genotypes) ought to include different stimulus waveforms. Importantly, reliable delivery of slow stimuli like 15 second-long ramps requires the stability demonstrated for the photostimulator in FIG. 3B; photostimulus ramps cannot be satisfactorily delivered by handheld fiber optics.

FIG. 8A and 8B show the added benefit of having a clear view of the mouse from below to collect data about non-reflexive behaviors for correlation with the preceding reflexive response, in which:

FIG. 8A shows the time spent licking (in the left graph) or time spent guarding (in the right graph) during the post-stimulus period (2-minute duration+time remaining in ramp after withdrawal) plotted against withdrawal latency on the corresponding trial. Each point shows data from a single trial; data were collected from 5 TRPV1-ChR2 mice tested with 15 second-long optogenetic ramps. Lines show linear regressions; p values on graph summarize the strength of correlations: slow withdrawals are more likely to be followed by licking of the paw, whereas fast withdrawals are more likely to be followed by guarding, where the paw is lifted and clenched.

FIG. 8B reports the same data as in FIG. 8A, but shows the time spent guarding plotted against the time spent licking on a trial-by-trial basis. The size of each dot represents the withdrawal latency on that trial. The distribution of points shows that mice either guard or lick, but rarely combine the two behaviors. There is a strong tendency for pain researchers to assess evoked reflexes or spontaneous (i.e., non-reflexive) “pain” behaviors; these data show the value of assessing both on a trial-by-trial basis and demonstrate the feasibility of doing this with our device.

FIG. 9A to 9C shows the ability of the photostimulator to measure heat-evoked withdrawal and pain using radiant heating with an IR laser, in which:

FIG. 9A shows the withdrawal latency to equivalent radiant heating at baseline (left, black) and after injecting 0.5% capsaicin into the hind paw (right, gray). Eight mice were each tested with 3 trials under baseline conditions and with another 3-5 trials after receiving capsaicin. Dotted lines show the average response for each mouse before and after capsaicin. Bars show the group average (±SEM). Latency dropped from 7.18±0.74 s at baseline to 3.88±0.49 s after capsaicin (t₇=4.64, p=0.002, paired t-test), consistent with past work showing that capsaicin causes thermal hypersensitivity. Thermal stimulation with the IR laser was automatically terminated after automatic detection of withdrawal.

FIG. 9B shows frames captured from video illustrating non-reflexive behaviors during the post-stimulation period. Left panel labeled “a” shows licking. Middle panel labeled “b” shows guarding, in which the paw is lifted off the platform and held in a clenched position. Right panel labeled “c” shows flinching. Histogram on the right summarizes the proportion of trials in which these behaviors where evident after radiant heating; black bars show trials under baseline conditions; gray bars show trials after capsaicin injection. All behaviors were significantly more common after capsaicin; p values on graph show results of χ² tests. All three behaviors were rare in the pre-stimulus period for both baseline and +capsaicin conditions.

FIG. 9C shows correlation between guarding and withdrawal latency. Specifically, the occurrence of guarding on a given trial (yes/no) is plotted against withdrawal latency on that trial. Left graph shows data under baseline conditions. Right graph shows the same analysis after injecting capsaicin. Thick curves show logistic regressions; shading shows 95% prediction interval. According to logistic regression, guarding was significantly more likely after shorter-latency responses in +capsaicin condition (p=0.0098) but not in baseline condition (p=0.605). Like in FIG. 8, these results illustrate the value of quantifying evoked (reflexive) and spontaneous (non-reflexive) behaviors together; for instance, increased licking after short-latency responses in the +capsaicin condition suggests that thermal stimulation is indeed more painful and that increased pain is paralleled by reduction of withdrawal latency. Standard radiant heat testing (Hargreaves) devices are not amenable to such analysis because the IR light source obscures the view of the animal from below.

FIG. 10A to 10C shows measurement of touch-evoked withdrawal using a length-controlled mechanostimulator with simultaneous force measurement, in which:

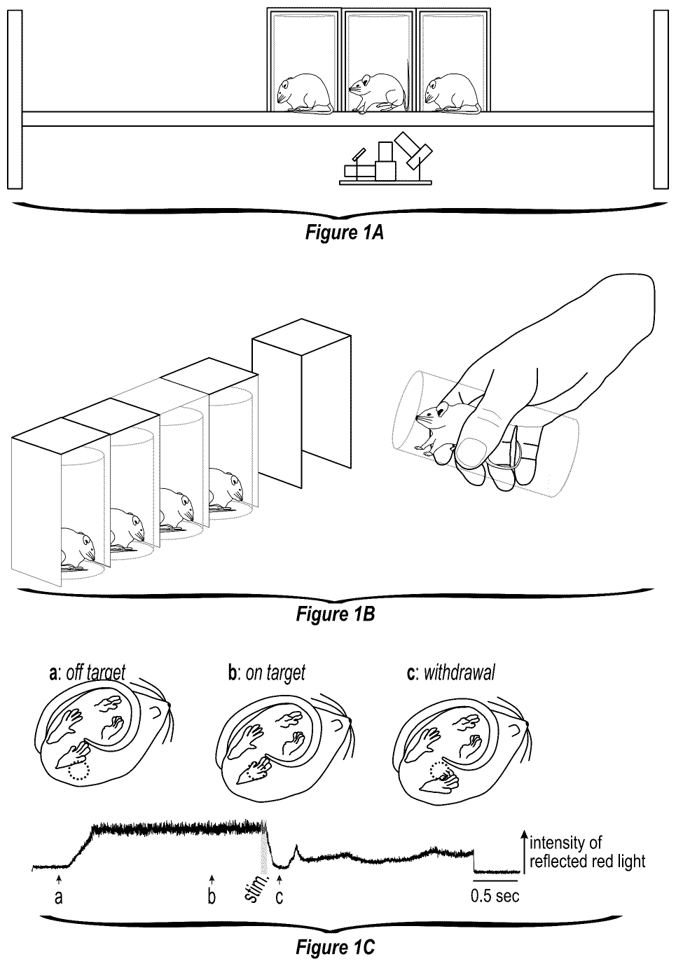

FIG. 10A shows the mechanostimulator with the mechanostimulus controller mounted on a pedestal so that it is positioned just below the platform, but to the side, so that it does not obscure the view of the mouse. The mechanostimulus controller (a 300C-I dual-mode indenter from Aurora Scientific) is positioned so that its indenter arm can be raised between the bars of the metal grate floor to press against the target paw. In the version depicted here, components are mounted on motorized linear actuators, which control aiming. In a manually aimed version of the device, components are mounted on a breadboard and slid horizontally by hand, as shown for the photostimulator in FIG. 1A. The device depicted here acts like an electronic von Frey, to “poke” the paw with a blunt probe. The tip of the indenter arm can also be sharp, to deliver a needle prick. Alternatively, the indenter arm can be modified by addition of a Peltier device to deliver hot or cold stimuli by contact (as opposed to contactless, radiant heating), or to apply acetone to induce cooling or other chemical agents. Instead of an indenter arm, a rotary brush can be mounted just below the metal grate floor to deliver dynamic touch stimuli. All of these variants, whether to deliver mechanical, thermal or chemical stimulation, are controlled and monitored by computer and are referred to here as a “mechanostimulator” variant for distinction from the “photostimulator” variant; the former applies stimulation by contact whereas the latter which relies entirely on light. The two variants can be combined in a single hybrid device.

FIG. 10B shows how precise mechanostimulation is delivered. The bottom trace shows how the height of the indenter arm plotted against time as it is ramped up by computer command. This degree of control far exceeds what is possible with handheld von Frey filaments, including typical electronic von Frey devices. For instance, the indenter arm height can be raised at different speeds or moved according to other waveforms. The top trace shows the force exerted on the paw by the indenter arm, which is measured by the mechanostimulator at 1 KHz. Threshold for each trial is taken as the force immediately preceding withdrawal. Arrows marked a-c indicate times at which frames were extracted from simultaneously recorded video.

FIG. 10C shows frames extracted from the video used for aiming at 100 fps (top panels) and from high-speed video at 1000 fps (bottom panels). Dotted ellipses in bottom panels highlight the target paw. Left panels labeled “a” show the indenter arm aimed at the target paw before initiating stimulation. Middle panels labeled “b” show the indenter arm pushing against the paw. Right panels labeled “c” show the paw withdrawn from the indenter arm; the paw no longer visible inside the dotted ellipse.

FIG. 11A to 11E show the motorized version of the device and its performance, in which:

FIG. 11A shows the how the components of the systems are configured, like in FIG. 1A, but now with the photostimulator mounted on motorized linear actuators that control horizontal movements left-right (in x-axis) and forward-back (in y-axis). Other variations of the device have the stimulator mounted on three actuators, where two fine actuators control movement in x and y around a single mouse, and one long actuator moves the fine actuators between individual mice. Mice are positioned at equally spaced distances left to right across the platform. It is also possible to have multiple rows, arranged in forward and rear positions. Different positions can have the same or different platform flooring, namely plexiglass for photostimulation and metal grate for mechanostimulation and other stimulation modes involves direct contact.

FIG. 11B shows a top-down view of the parts described in FIG. 11A.

FIG. 11C shows how automated aiming is implemented by illustration of the mouse before and after aiming is complete. Prior to testing, a neural network is trained with the help of software like DeepLabCut to recognize the target paw as well as other key points (other paws, snout, tail base, etc.) for pose estimation. During testing, the video is fed to computer, which uses the pre-trained neural network to recognize the target paw in real-time (i.e., faster than video frames update). Left image labeled “a” shows paw before aiming is complete. Position of the target paw as identified by the neural network is marked with O. Arrows show the spatial separation of the target paw from the crosshairs (i.e., where photostimulation will be delivered). The spatial separation is subdivided into x- and y-error signals. Guided by those error signals, the computer drives the actuators to reposition the stimulator and minimize the error signals. Actuator speed is proportional to the error signal amplitude. The right image labeled “b” shows the circle marking the target paw now centered in the crosshairs, meaning aiming is successful. Aiming is complete when the circle center is positioned <3 pixels from the crosshair center. At that point, testing proceeds as explained in FIG. 5D.

FIG. 11D shows a flow chart of the processes controlling aiming and stimulation. In each video frame, the target paw is inferred using AI and its position is used to calculate error, as explained in FIG. 11C. The stimulator is moved by motorized actuators to reduce error, and this process continues until the error is within the tolerance level. At that point, red light is initiated and a timer is started. If the error signal remains within the tolerance for a minimum acceptable interval, indicating that the mouse is stationary, photostimulation with blue or IR light is initiated. If the mouse moves during this pre-stimulus period, the stimulation sequence is aborted and aiming resumes. Withdrawal is automatically detected and withdrawal latency measured from the reflectance signal, as explained in FIG. 5. Photostimulation is automatically terminated when withdrawal is detected, or when the full duration of the stimulus is complete, whichever occurs first.

The video collected for aiming is automatically recorded and can be used to later analyze non-evoked behaviors in the pre- or post-stimulus periods, as illustrated in FIG. 8 and FIGS. 9B and 9C. Variations in the stimulus control are possible; for example, by automatically detecting various postures, stimulus initiation could be made contingent on the mouse adopting a desired posture, or stimulus initiation could be prohibited when the mouse assumes an undesired posture (e.g., when rearing). In the fully automated setting, the stimulator automatically moves to the next mouse after completing a testing sequence, and proceeds to test the next mouse. The same automated video-based aiming process is used for the mechanostimulator variant of the device. Although the indenter arm and metal bars partially obscure the view of the mouse from below, a neural network trained with the obstacles present can learn to recognize the target paw and successfully effect automated aiming.

FIG. 11E shows that automated aiming can be very precise. Light-on-target was measured in the same way as described in FIG. 3. For automated aiming of the photostimulator, a neural network was trained to recognize the paw-shaped cut out placed over the photodiode. The photostimulator was automatically aimed at the paw-shaped cut out and delivered pulses 10 times; the photostimulator was forced to re-aim after each pulse. The manually aimed photostimulator data from FIG. 3C, which includes 10 trials per tester from 5 testers, for a total of 50 trials, was used for comparison. Deviation from the mean dropped from 11.0±0.4 μW (mean±SEM) for manual aiming (shown in gray) to 3.7±0.4 μW for automated aiming (shown in black). This drop is significant (t₅₈=3.03, p=0.004, unpaired t-test), indicating that automated aiming is significantly more precise than manual aiming.

FIG. 12A and 12B show the graphical user interface (GUI) and spreadsheet associated with the software used for stimulator control, in which:

FIG. 12A shows the GUI with key components labeled. In this embodiment, there are positions for 24 mice on the platform, organized in two rows of 12. The stimulator moves directly to the position clicked on in the GUI; the position is recorded as metadata for each trial. During the aiming and stimulation sequence, one can monitor progress with the live video and with the simultaneously updated stimulus and reflectance signals. Parameters are set at the bottom right. The user can switch between automated aiming and aiming via joystick or key press.

FIG. 12B shows a screenshot of the spreadsheet to which data and metadata for all trials are automatically recorded. Metadata include the exact date and time of each trial, the mouse identity (position), and all stimulus parameters. The withdrawal latency is automatically measured and recorded, along with the raw stimulation and reflectance data, which are provided as linked graphs. Video for all trials also saved and conveniently provided as links. The systematized curation of data and metadata thus achieved is critical for creation of large data sets.

FIG. 13 shows different behaviors automatically classified from video. Each image shows a frame extracted from the video to illustrate a different behavior indicated by the label. Various algorithms have been developed to automatically segment behaviors using supervised or unsupervised learning. Gray shaded dots show latent space embeddings of behavioral state determined at regular intervals from the video. This example classification is provided to show how analysis of spontaneous behaviors can be automated using the sort of video our device collects. In future, real-time classification of behaviors can be used to implement closed loop control of stimulation, as discussed under FIG. 11B.

### DETAILED DESCRIPTION

The present disclosure relates to an apparatus for automated pain testing in laboratory rodents and is illustrated below with respect to mice, however it will be understood the present system and method is applicable to rodents in general. Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in this specification including claims, the terms, “comprises” and “comprising” and variations thereof mean the specified features, steps, or components are included. These terms are not to be interpreted to exclude the presence of other features, steps, or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures, or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

### Detailed Description of Methods

### Animals

All procedures were approved by the Animal Care Committee at The Hospital for Sick Children and were conducted in accordance with guidelines from the Canadian Council on Animal Care. To express ChR2 selectively in different types of primary somatosensory afferents, we used Ai32(RCL-ChR2(H134R)/EYFP) mice (JAX:024109), which express the H134R variant of ChR2 in cells expressing Cre recombinase. These were crossed with advillin^{Cre} mice (kindly provided by Fan Wang) to express ChR2 in all sensory afferents, TRPV1^{Cre} mice (JAX:017769) to express ChR2 in TRPV1-lineage neurons, or Nav1.8^{Cre} mice (kindly provided by Rohini Kuner) to express ChR2 in nociceptors. 8-16 week old male or female mice were acclimated to their testing chambers for 1 hr on the day before the first day of testing, and each day for 1 hr prior to the start of testing. Sex differences were not observed and data were therefore pooled.

### Platform and Enclosures

The platform and animal enclosures were custom made. Except when testing mechanical or other contact-based stimuli, the platform is 3 mm-thick clear Plexiglass mounted on 20×20 mm aluminum rails, adjusted to a fixed height above the stimulator. For mechanostimulation, plexiglass was replaced with a metal grate comprising stainless steel rods. For enclosures, clear Plexiglass tubes (outer diameter=65 mm, thickness=2 mm) cut in 12.5 cm lengths were used in conjunction with opaque white 3-D printed cubicle. The same tube used to transfer a mouse from its home cage is placed on the platform vertically and slid into a cubicle for testing (see FIG. 1B). A notch cut into the base of each tube allows the experimenter to deliver a food reward, to poke the mouse (to wake or orient it), or to clean feces or urine from the platform if required. Each cubicle is 3-D printed and contains internal magnets that allow cubicles to be easily combined. Keeping the mice at fixed distances from each other is important for automated testing, where the stimulator is automatically translated a fixed distance when testing consecutive mice. The system is compatible with other enclosure designs; for example, to view the mouse in profile during high-speed video, we used a narrow rectangular chamber with clear walls on the front and left side (with a notch under the latter) and opaque walls at the rear and right side. In some cases, a mirror was placed at a 45° angle near the left wall to simultaneously capture a front view of the mouse.

### Photostimulator

Collimated light from a red (625 nm) LED and blue (455 nm) LED is combined using a 550 nm cut-on dichroic mirror. Blue light is attenuated with a neutral density filter. This beam is combined with IR light from a 980 nm solid laser using a 900 nm cut-on dichroic mirror. The IR beam is expanded to fill the back of the focusing lens. The common light path is reflected upward with a mirror and focused to a spot 5 mm in diameter on the platform above. The surface area of the spot is ˜20 mm²; photostimulus power values should be divided by this number to convert to light density (irradiance). Red light reflected off the mouse paw is collected by a photodetector through a 630 nm notch filter. All light sources are controlled by computer via appropriate drivers and a 1401 DAQ (Cambridge Electronic Design) using Spike2 (Cambridge Electronic Design) or custom software written in Python. The photodetector samples at 1 KHz with the same DAQ, thus synchronizing stimulation and withdrawal measurement.

### Mechanostimulator

Computer-controlled mechanostimulation was implemented using a 300C-I dual-mode indenter (Aurora Scientific), which can control and measure both force and length (height). Our software controls height in the same way LED/laser intensity is controlled for photostimulation. The exerted force is simultaneously measured at 1 KHz and recorded to computer. Because withdrawal is evident from changes in measured force, additional signals (e.g., reflectance, video) are not required for latency measurements.

### Manual Aiming

A camera provides video of the mouse from below (substage). Video is used for aiming with the help of visual feedback using the red light, which is turned on prior to photostimulation with blue or IR light. Red light is not used to help aim the mechanostimulator since positioning of the indenter arm relative to the paw is obvious from video. A near-IR light source is useful to improve lighting during high-speed video. In the manual version of the device, the device is slid by hand; leveling screws at the four corners of the breadboard have a plastic cap for smooth sliding. In the motorized version (described below), the breadboard it attached to linear actuators (TBI Motion) via 3-D printed connectors.

Comparison with Handheld Fiber Optic

To measure the effects of aiming on stimulus delivery, volunteer testers were instructed to use a fiber optic (multimode fiber optic patch cable, 1000 mm diameter core, NA=0.48, SMA endings attached to 455 nm fiber-couple LED, Thorlabs) to apply a photostimulus to an s170C photodiode attached to a PM100D optical power meter (Thorlabs). The same photostimulus power was used for all trials, by all testers. The photodiode was covered with a paw-shaped cut out and placed face down on the plexiglass platform to simulate aiming at a real paw standing on the platform. The PM100D output was connected to a Power1401 data acquisition interface (Cambridge Electronic Design), sampling at 1 KHz. The Power1401 was also used to deliver command voltages to the LEDD1B LED driver (Thorlabs).

### Automated Withdrawal Detection and Latency Measurement

Paw withdrawal from photostimulation is detected and its latency measured from the red reflectance signal using custom code written in Python. Red light is initiated prior to photostimulation with blue or IR light. Baseline reflectance is measured over a defined period (0.5-2 s) preceding photostimulus onset. A running average across a 27 ms-wide window was used to remove noise. Withdrawal latency was defined as time elapsed from photostimulus onset until the reflectance signal dropped below a threshold defined as 2 mV below baseline; the signal needed to remain below threshold for >20 ms to qualify as a response, but latency was calculated based on the start of that period. The 2 mV threshold value was chosen based on pilot experiments and then applied unchanged in all subsequent testing. Latencies thus extracted from the reflectance signal were compared to latency values extracted from high-speed video of the same withdrawal. In the latter case, paw height was extracted from video (see below) using DeepLabCut; latency was taken as the time taken for paw height to rise 6 pixels above baseline, defined as the mean height over the 0.5 s epoch preceding photostimulus onset. All latency measurements reported in the manuscript are based on automated reflectance-based measurements unless otherwise indicated.

### Validation of Latency Measurements Using High-Speed Video

High-speed video was collected with a Chronos 1.4 camera (Krontech) using a Computar 12.5-75 mm f/1.2 lens sampling at 1000 fps. To synchronize video with stimulation, the camera was triggered with digital pulses sent from the DAQ. Videos

were compressed using H.264. Video was analyzed using DeepLabCut to label the hind paw in sample frames and train a deep neural network to recognize the paw. This returned paw trajectories which were analyzed using custom code written in Python. Withdrawal latency measured from paw height were compared trial-by-trial with latency measured from the reflectance signal.

### Pose Estimation

DeepLabCut-Live was used to track mouse pose from substage video. While we stimulated only the left hind paw, networks were trained to recognize the snout, front paws, hind paws, and tail base. The extra keypoints were intended to force the network to assume weights that would represent orientation well, and distinguish between the left and right paws. Paws were not labelled when guarded (turned such that the plantar surface was not visible) so that the network would not recognize the paw, and therefore not apply a stimulus. To train the neural network, we collected one video with 9 mice on the photostimulator platform and panned the camera around under the mice using the linear actuators, collecting 9 minutes of video. 500 frames were labelled and 95% were used for training a ResNet-50-based neural network with default parameters for 200,000 iterations. We validated on one shuffle and found a test error of 17.41 pixels (px) and train error of 2.62 px. These error values represent multiple keypoints; test error specifically related to the target hind paw is much lower (3.33 px). The image size was 640×480. Importantly, the hindpaws were not labelled when the paws were turned in a guarding. This meant that the paws would not be recognized unless placed flat on the platform, and that stimulation would only occur when the paws were correctly oriented. Training was done on a 32 GB NVDIA Tesla V100 GPU, while live inference for aiming was done on a 3 GB NVIDIA Quadro K4000 or NVIDIA Geforce RTX 4070 Ti (see below). Different networks were required for different applications. To analyze paw withdrawal height, a separate neural network was trained using high-speed video of the mice in profile. DeepLabCut was used with the same parameters as above, training on 580 frames of high-speed video with a 1008×500 resolution. Test error=5.05 px; train error=2.32 px. For automated mechanical stimulation, another neural network was trained that could recognize the mouse on a metal grate. Again, the same parameters were used for DeepLabCut, but training on 100 frames with a 1280×800 resolution. Test error=19.15 px; train error=1.82 px. To validate photostimulus reliability with automated aiming, a network was trained to recognize a paw-shaped cut out covering a photodiode (see above). The same parameters were used as mentioned above, training on 200 frames with a 640×480 resolution, while labeling the center of the paw-shaped cut out. This yielded a test error of 2.43 px and a train error or 2.1 px.

### Automated Aiming

The substage camera was aligned with the linear actuators such that movements in the x- or y-directions in the video feed could be independently initiated be the x- or y-linear actuators. The camera was also positioned such that the center of the frame was aligned with the photostimulus. x- and y-error signals were then calculated by taking the distances from the DeepLabCut-live-based pose estimates for the target paw to the center of the frame (see FIG. 11C). Inference for one frame on an NVIDIA Geforce RTX 4070 Ti could be completed in <20 ms, which is less than the duration of each frame for standard-rate video ( 1/30 fp =33 ms). The x- and y-linear actuators were then independently driven with signals that were proportional to the error signals. Importantly, the proportionality of the actuator speed to the error signal reduced the velocities of the linear actuators as they approached the target, preventing overshoot. Once the target was centered in the frame within a tolerance of 3 px, a timer begins for a user-defined amount of time (typically 1-2 s) before the stimulus is initiated. This delay assures that the mouse is immobile when initiating stimulation. If the target paw moves within the pre-stimulus interval, aiming is re-initiated and, once aligned, the timer is restarted. A separate timer can be set to stop this sequence and move to the next mouse for cases where a certain mouse is too active to stimulate reliably.

### Behavior Extraction From Substage Video

Substage video was saved and compressed using H.264. DeepLabCut was used to identify the nose, left fore paw, right fore paw, left hind paw, right hind paw, and tail base. These key points were then fed into the VAME framework using default parameters and 10 clusters to extract complex behaviors [66].

### Description of Algometer Device and Its Capabilities

### Configuration of Equipment

Mice (or other laboratory rodents) are kept individually in enclosures on a clear platform with the stimulator underneath (FIG. 1A). A wire grate floor is used when testing mechanical stimuli (see FIG. 10C). The mouse is transferred from its home cage in a clear plexiglass tube, which is then turned vertically and slid into an opaque ceilinged cubicle for testing (FIG. 1B). Tube/tunnel handling is less stressful than other handling methods [33-35] and using separate tubes facilitates addition or removal of individual mice from the set of cubicles. Other cubicles can be used, as appropriate for specific experiments. Video of the mouse from below is used for aiming with the help of red light which, like the laser scope on a gun, identifies the photostimulation zone before photostimulation is initiated (FIG. 1C).

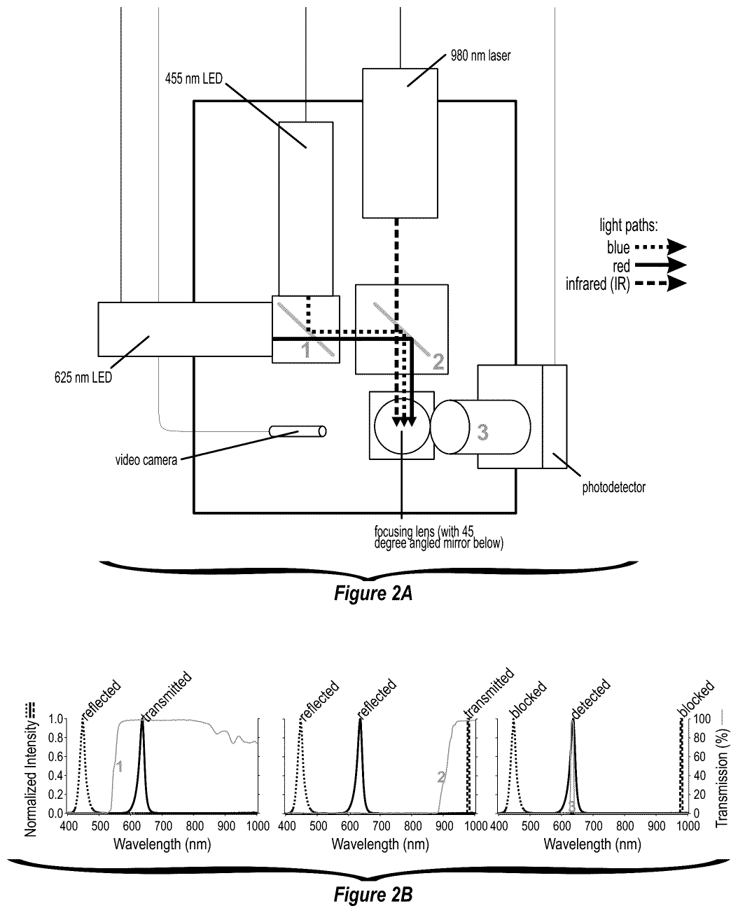

FIG. 2A shows the stimulator viewed from above. Blue light for optogenetic activation using ChR2, IR light for thermal stimulation (radiant heating), and red light for aiming and response measurement are combined into a single beam using dichroic mirrors (FIG. 2B). Other light sources can be included, such as green for optogenetic inhibition using Archaerhodopsin, depending on the experiment. The beam is directed vertically and focused to a spot approximately 5 mm in diameter on the platform above. An adjacent camera collects video from below (substage) while a photodetector measures red light reflected off the paw. The stimulator is translated manually or by motorized actuators. For mechanical stimulation, a computer-controlled indenter is positioned below the wire grate floor (see FIG. 10A) but manual or motorized/automated aiming is the same as described above.

Since all wavelengths converge on the same spot, red light is turned on prior to initiating photostimulation (with blue or IR light) to verify where photostimuli will hit, thus providing visual feedback to optimize aiming (FIG. 1C). Rodents are typically

assumed not to see red light [36]; though some evidence contradicts this [37, 38], we never observed any behavioral response to red light (little of which likely reaches the eyes when applied to the hind paw), suggesting that the aiming phase does not provide mice any visual cue about the forthcoming photostimulus. Reflectance of red light off the paw is measured by the adjacent photodetector. Maximization of the reflectance signal can be used to optimize aiming. The reflectance signal is stable while the paw and stimulator are immobile but changes when the paw is withdrawn, thus enabling measurement of withdrawal latency (see FIG. 5). Though too slow to accurately measure fast withdrawals, standard video recordings allows one to visually rule out gross errors in reflectance-based latency measurements and also enables off-line assessment of slower behaviors like guarding and licking (see FIGS. 8 and FIGS. 9B and 9C).

### Reproducible Photostimulation

Unaccounted for variations in stimulation fundamentally limit the precision with which stimulus-response relationships can be characterized. LEDs and lasers offer stable light sources but the amount of light hitting a target can vary over time or across trials depending on the accuracy and precision of aiming. When applying light by hand-held fiber optic (as typically done for transcutaneous optogenetic stimulation), stability of the tester and differences in aiming technique across testers are important. To gauge the importance of aiming, we measured how the amount of light hitting a target depended on the fiber optic's positioning in the x-y plane and its distance (z) below the platform. Light was delivered through a paw-shaped cut out to a photodiode facing downward on the platform (to simulate stimulation of a mouse paw) while controlling fiber optic position with linear actuators. FIG. 3A shows that light delivery is sensitive to positioning in all three axes, especially in z (because light rays diverge from the fiber optic tip).

To explore the practical consequences of this, we measured light delivery while 13 testers applied a 10 s-long photostimulus by handheld fiber optic (FIG. 3B, gray traces). The signal-to-noise ratio (SNR=mean²/SD²) of 23.5±2.0 dB (group mean±SEM) was significantly less than the 55.6 dB obtained with the stimulator (black trace) (t₁₂=15.8, p<0.001, one sample t-test). The mean stimulus intensity also differed across testers with an inter-tester coefficient of variation (CV=SD/mean) of 18.8%, which is even larger than the average intra-tester CV of 8.9%. In other words, during a sustained photostimulus, temporal variations in light-on-target arise from each tester's instability, but this variability is compounded by differences in aiming technique across testers.

The same issues affect short (pulsed) stimuli but manifest as trial-to-trial variations. To measure variability across trials, five testers used a handheld fiber optic or the photostimulator to deliver ten 100 ms-long pulses to a photodiode (FIG. 3C); each pulse was triggered independently. Trial-to-trial deviation of each tester from their individual mean dropped from 26.3±3.0 mW (mean±SEM) with the handheld fiber optic to 6.4±0.7 mW with the photostimulator (t₉₈=6.42, p<0.001, unpaired t-test), which represents a 75.6% reduction in intra-tester variance. Deviation of each tester from the group mean fell from 44.5±18.2 mW with the fiber optic to 9.7±2.0 mW with the photostimulator (t₈=1.94, p=0.093), which represents a 78.2% reduction in inter-tester variance. In other words, using the photostimulator increased reproducibility of stimulation across testers and within each tester. This is because the photostimulator's distance below the platform is fixed and light rays converge, and because aiming is improved by using video and visual feedback from red light.

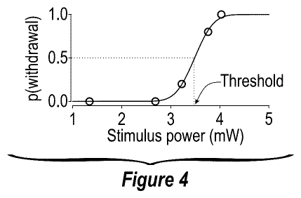

Even if stimulation is reproducible, behavior is still variable, especially in response to weak stimuli. Indeed, threshold is defined as the stimulus intensity at which withdrawal occurs on 50% of trials. FIG. 4 shows determination of optogenetic threshold. Reliable aiming combined with precisely controllable LEDs (whose output can be varied in small increments over a broad range) allows one to measure threshold and characterize the broader stimulus-response relationship, assuming responses can be identified clearly and measured precisely.

### Precise Response Measurement

High-speed video is the gold standard for measuring fast behaviors, but acquiring and analyzing those data is complicated and costly. We sought to replace high-speed video by detecting changes in the amount of red light reflected off the paw (see FIG. 1C) using a low-cost photodetector. To validate our method, response latency was determined from high-speed video for comparison with latency determined from the reflectance signal on the same trials (FIG. 5A). The stimulated

paw was identified (dot in sample frames) using DeepLabCut and paw height was measured from each frame. Latency was determined independently for each signal based on the time taken for that signal to cross a threshold defined as an absolute change from its pre-stimulus baseline; after its determination in pilot tests, the same threshold value was applied for all subsequent measurements. Each data point in FIG. 5B shows the reflectance-based and height-based latency measurement from a single trial plotted relative to one another; data are from 6 mice given 100 ms-long blue pulses with intensities spanning a broad range. Based on visual inspection of raw data, the rate of gross errors is low (<2%) for each method (FIG. 5B). The regression line (gray dotted line, slope=1.007) follows the equivalence line (black dashed line, slope=1). Transforming these data to a Bland-Altman plot (FIG. 5C) shows there is no fixed bias and that any proportional bias is inconsequential. Beyond avoiding an expensive high-speed camera and the challenges of filming the mouse in profile to assess paw height, the reflectance signal can be processed in real-time to enable closed-loop termination of photostimuli (i.e.

stimulation is terminated automatically once paw withdrawal is detected). High-speed video is not, therefore, essential for precise latency measurements but can provide additional information about the fast response [26].

### Measuring Input-Output Relationships

Minimizing variability in stimulus delivery and response measurement maximizes discrimination of small biological differences; indeed, an input-output relationship is obscured by poorly controlled input or poorly measured output adding noise respectively to the x- and y-positions of constituent data points. To explore how well our device reveals stimulus-dependent variations in withdrawal latency, we titrated the intensity of 100 ms-long pulses of blue light to determine the optogenetic threshold in each of 10 mice. Then, using intensities at defined increments relative to each mouse's threshold, we measured withdrawal latency as a function of photostimulus intensity (FIG. 6). Responses evoked by near (peri)-threshold intensities occurred with long latencies (>75 ms) but small increments in intensity evoked responses whose latencies were bimodally distributed. Further increments in intensity caused a complete switch to short-latency (<75 ms) responses. The proportion of slow and fast responses varied significantly with photostimulus intensity (χ²=105.0, p<0.0001), [40]. The data disclosed herein shows that response latencies within each group decreased with increasing photostimulus intensity, as described by linear regressions on log-transformed data.

### Additional Photostimulus Waveforms

To date, all reports of withdrawal from transcutaneous optogenetic stimulation used a pulse of blue light or a train of pulses [30, 40-51], with one exception, which

used sustained light to activate keratinocytes [52]. Yet different photomstimulus waveforms may reveal different information about the neural control of behavior, and so testing with different waveforms will provide greater information than testing with any one waveform. Therefore, capitalizing on the stability of our stimulator (see FIG. 3B), we tested slowly ramped photostimuli (and pulses) in two transgenic mouse lines: Advillin-ChR2 mice express ChR2 in all somatosensory afferents [53] whereas Nav1.8-ChR2 mice express ChR2 selectively in nociceptors [54, 55]. Responses to ramps differed dramatically between genotypes (FIG. 7A), with all Nav1.8-ChR2 mice responding with a mean±SD latency of 1.1±0.2 s whereas Advillin-ChR2 mice responded with much longer latencies on some trials, resulting in distinctly skewed distribution. The difference was mostly due to intra-mouse variability, with individual Advillin-ChR2 mice responding with a broad range of latencies rather than some mice being consistently slow and others being consistently fast. By comparison, both genotypes exhibited a similar bimodal latency distribution when tested with pulses (FIG. 7B). Notably, latencies are nearly three orders of magnitude slower for ramp-evoked responses than for pulse-evoked responses, meaning “slow” pulse-evoked responses are still much faster than “fast” ramp-evoked responses. Our goal here was not to compare pulse and ramp stimuli but, rather, to show that one stimulus waveform might reveal differences (e.g. between genotypes) that are not revealed by other waveforms, attesting to the value of testing with different stimulus waveforms in addition to different stimulus intensities and modalities.

### Analysis of Associated, Non-Reflexive Behaviors

The substage camera used for aiming provides a video record from which behaviors beyond reflex withdrawal can be analyzed. Non-evoked behaviors like licking, guarding, or flinching of the paw are typically interpreted as signs of ongoing or spontaneous pain. Though not directly evoked by stimulation the same way as a reflex, it is valuable to consider whether stimulation modulates the probability of those behaviors, as this could be interpreted to mean a stimulus causes lasting pain. Capitalizing on the video record provided by the substage video, we analyzed spontaneous behaviors following optogenetic ramp stimuli like those reported in FIG. 7. Notably, the high intra-mouse variability in withdrawal latency in response to ramps affords an ideal opportunity to test if short- or long-latency withdrawals in the same mouse are more or less painful. Plotting the amount of time spent licking or guarding (during a ˜2-minute post-stimulus period) against withdrawal latency (FIG. 8A) revealed that long-latency withdrawals were associated with significantly more licking (T₅₅=5.06, p=4.96×10⁻⁶, one-sample t-test on slope) but not more guarding (T₅₅=−1.62, p=0.111). This suggests that failure to withdraw promptly—for reasons that remain unclear—results in the stimulus causing more pain, as inferred from licking. Interestingly, plotting the time spent licking against time spent guarding on a trial-by-trial basis shows that mice tend to exhibit one or the other behavior on a given trial (FIG. 8B). Interpretations warrant caution but automated analysis can expedite and help standardize future investigation along these lines.

Radiant Heat Stimulation

Despite focusing hitherto on optogenetic stimuli, our device can deliver other, more conventional stimuli and automatically measure withdrawal therefrom. Radiant heat is applied with an IR laser. Laser intensity was adjusted in pilot experiments to evoke withdrawal after ˜8 sec (like in a standard Hargreaves test). Test stimuli were automatically terminated upon detection of paw withdrawal (see above) or after a 20 s cutoff. Withdrawal latency was significantly reduced after injecting 0.5% capsaicin into the hind paw (FIG. 9A). Video of withdrawals revealed that thermal stimulation triggered significantly more licking, guarding, and flinching after capsaicin (FIG. 9B). By plotting the occurrence or absence of these non-reflexive behaviors against latency of the preceding withdrawal, logistic regression reveals whether probability of a behavior is correlated with withdrawal latency. For example, guarding was not correlated with withdrawal latency under baseline conditions but, after capsaicin, was significantly more likely after short-latency withdrawals (FIG. 9C) (logistic regression, p=0.605 based on 22 baseline trials vs p=0.00985 based on 32 +capsaicin trials). One may cautiously interpret this to mean that capsaicin causes heat to be perceived as more painful, triggering faster withdrawal, whereas short-latency responses occasionally occur under baseline conditions but not because certain trials are more painful than other trials, providing clues as to where variability arises [56].

Notably, standard Hargreaves (radiant heating) devices are not compatible with substage video because of the close proximity of the light source to the paw. This precludes or at least significantly complicates video-based analysis of non-evoked behaviors. Furthermore, without a clear view of the target paw, manual aiming is less precise and automated aiming is infeasible.

### Mechanical Stimulation and Other Stimuli Involving Physical Contact With Paw

Mechanical stimulation is applied with a computer-controlled, force-feedback indenter (FIG. 10A). With the mouse positioned on metal grate floor (instead of plexiglass), the indenter tip is aimed via substage video before the indenter arm is raised at a fixed rate (FIG. 10B). Withdrawal is evident from the rapid drop in force as sensed by the indenter (at 1 KHz). Mechanical threshold is taken as the peak force immediately prior to withdrawal. Video from two different angles, including high-speed video at 1000 fps, confirms that the drop in force corresponds to when the paw is withdrawn from the indenter arm (FIG. 10C). Because withdrawal is evident from force measurements measured at 1 KHz, a reflectance signal using red light is not required. In the example response illustrate in FIG. 10, the indenter arm had a blunt tip; however, the tip can be modified to deliver different types of stimuli including need prick, contact heat or cold, and chemical agents. The indenter arm can also be replaced, for instance, with a rotary brush to provide dynamic mechanical stimuli. All of these contact-based stimuli require use of the metal grate platform, instead of plexiglass, to provide direct access to the paw.

### Fully Automated Testing

The photostimulator was mounted on linear actuators (FIG. 11A and 11B) so that aiming could be controlled remotely by joystick or key press (i.e., without the tester operating in close proximity to the mice) or automatically using AI. For the latter, a neural network was trained using DeepLabCut to recognize the paws and other points on the mouse viewed from below. The computer is then fed the substage

video stream and DeepLabCut-Live uses the trained network to locate the target paw. Error signals are calculated from the target paw location and minimization of those error signals is used to position the photostimulator (to within 3 pixels) so that the target paw is positioned in the crosshairs for stimulation (FIG. 11C). Stimulation is automatically initiated once the paw has remained stationary for a minimum period (FIG. 11D) and is automatically terminated upon detection of paw withdrawal or after a pre-determined cut-off. Automated aiming delivered stimuli even more reproducibly than manual aiming with the same device (FIG. 11E). For mechanical stimulation, the metal grate floor partially obscures the mouse but automated aiming is still possible with an appropriately trained neural network.

After completing a trial, the device is shifted to the neighboring mouse. By interleaving trials, other mice are tested during the inter-stimulus interval required for each mouse, thus expediting the overall testing process. The order of testing can easily be randomized, which is difficult for an experimenter to keep track of, but is trivial for a computer. Stimulus parameters for the next trial can be automatically chosen using algorithms that factor in the presence or absence of responses on preceding trials in a given mouse. FIG. 12A shows the graphical user interface (GUI) of the software used to control the device and acquire data. Data (withdrawal latency, video) and meta-data (mouse identification, date, time, stimulus parameters) are automatically saved to a spreadsheet (FIG. 12B).

Non-reflexive behaviors like those analyzed in FIG. 8 and FIGS. 9B and 9C can also be detected and quantified automatically using AI (FIG. 13). Beyond expediting and helping standardize quantification of ongoing behaviors, automated classification in real-time can be integrated with withdrawal testing to implement closed-loop control so that stimuli are applied contingent on certain postures; for example, guarding and rearing can influence withdrawal latencies [40, 58], and stimulation should therefore be delayed until the mouse assumes a preferred posture. Overall, standardized high-throughput testing without the potential errors, systematic differences [19], and animal stress [59] associated with human testers is thus realized.

### Discussion

Capitalizing on identification of gene expression patterns to distinguish subtypes of somatosensory afferents [60], optogenetics affords an unprecedented opportunity to study somatosensory coding by activating or inhibiting specific afferent subtypes. Testing the behavioral response to synthetic activation patterns allows one to explore causal relationships, complementing efforts to characterize co-activation

patterns evoked by natural stimuli [61]. This does not require that optogenetic stimuli mimic natural, somatosensory stimuli. Optogenetic stimuli are patently unnatural—and the evoked sensations probably feel unnatural, like paresthesias evoked by electrical stimulation—but their ability to evoke behavior allows one to start inferring how they are perceived, and how those sensations relate to neural activation patterns. Doing this requires tight control of the stimulus and precise measurement of the response.

Technical advances have been made in delivering photostimuli to the CNS or peripheral nerves for optogenetic manipulations [62]. Transcutaneous stimuli are difficult to apply reproducibly to behaving animals. Sharif et al., [50] solved this by mounting the fiber optic to the head in order to stimulate the cheek, but a comparable solution is infeasible for stimulating paws. These technical challenges explain why past studies focused on whether mice responded to optogenetic stimulation, without carefully varying stimulus parameters or measuring subtler aspects of the response. Past studies have varied the number, rate, or intensity of pulses, but in the suprathreshold regime, with consequences for the amount of licking, jumping, or vocalization. To our knowledge, only one study [63] titrated the intensity of transcutaneous photostimuli to determine threshold, and another [45] titrated pulse duration and spot size. Moreover, scoring responses by eye, though still the norm for many tests, must be replaced with more objective, standardized metrics. Schorscher-Petcu et al. recently described a device that uses galvanometric mirrors to direct photostimuli and high-speed substage video to measure withdrawal [45]. Their device is very elegant but reliance on high-speed video to detect withdrawals likely precludes closed-loop control, and nor is their device fully automated or high-throughput. Our device delivers reproducible photostimuli and automatically measures withdrawals using the red-reflectance signal complemented by regular-speed substage video, which is also used for automated aiming.

Notably, non-painful stimuli may trigger withdrawal, which is to say that the threshold stimulus (or the probability of withdrawal) may not reflect painfulness [26]. In that respect, testing with stronger stimuli is also informative. The study by Browne et al. stands out for its use of high-speed video to thoroughly quantify responses to optogenetic stimulation [40]. Like us, they observed a bimodal distribution of

withdrawal latencies; however, they observed this despite using high-intensity pulses, most likely because their pulses were extremely brief (3 ms) and might, therefore, have activated afferents probabilistically. By varying the intensity of longer (100 ms) pulses, we observed that stronger stimuli evoke faster withdrawals, evident as a continuous shift in latency as well as a switching from long- to short-latency responses. A putative explanation for the bimodal latency distribution—consistent with Browne et al. [40] and with the double alarm system proposed by Plaghki et al. [64] based on different rates of heating—is that slow and fast responses are mediated by C- and A-fibers, respectively. Building from that, our data suggest that C-fibers are recruited first (i.e. by weaker photostimuli) and that slow responses speed up as more C-fibers get recruited, but a discontinuous “switch” to fast responses occurs once A-fibers get recruited, and fast responses speed up as more A-fibers are recruited. Further investigation is required, but that our device made possible the resolution of the stimulus-response relationship sufficiently to even pose such questions is notable.

Browne et al. also noted that the withdrawal response was not limited to the stimulated limb, and instead involved a more widespread motor response [40]. Though not quantified here, a widespread response was evident in the substage video and sometimes included vocalizing, facial grimacing, jumping, and orienting to the stimulus followed by licking, guarding or flinching of the stimulated paw. A complete analysis of each trial ought to consider not only the reflexive component (i.e. did withdrawal occur and how quickly), but also whether signs of discomfort were exhibited afterwards and for how long. Those signs are obvious when applying very strong stimuli but become harder to discern with near-threshold stimulation, which makes objective quantification all the more important. Regular-speed video, such as what we have installed on this device, is sufficient to capture all but the fast initiation of reflexive withdrawal (which can be measured by other means) and the bottom-up view is well suited for AI-based quantification of ongoing behaviors [65, 66]. We recommend that video be recorded for all trials if only to allow retrospective analysis of those data in the future. There has been an explosion of AI-based methods for extracting key points on animals [39, 67, 68] and algorithms for extracting higher-level animal behaviors from key point [65, 66, 69] or raw pixel [70] data, and rapid advances are likely to continue. Application of such tools is yielding impressive results [71]. Other hardware has been recently developed to facilitate such analysis but does not include stimulation capabilities [72]. By capturing video before and after stimuli, our device enables users to quantify behaviors in addition to measuring reflexive withdrawal using traditional metrics.

Withdrawal responses are known to be sensitive to posture and ongoing behavior at the time of stimulation [40, 58, 73]. Such differences may confound latency measurements but may also provide important information; either way, they should be accounted for. Waiting for each mouse to adopt a specific posture or behavioral state is onerous for human testers but is something that the neural network used by our device can be trained to do, with automated stimulation being made contingent on the mouse being in a certain state. But before that, to better understand the relationship between the mouse's pre-stimulus state and its subsequent stimulus-evoked withdrawal (and post-stimulus state), the state at the onset of stimulation could be classified from video and correlated with the evoked response on that trial. Other comparisons would also be informative, like correlating the pre- and post-stimulus states and treatment status. In short, more data can be acquired and more thoroughly analyzed than is typically done in current protocols; others have also advocated for this [25-27]. More comprehensive analysis need not entail expensive equipment or reduced throughput.

Nearly all past studies involving transcutaneous optogenetic stimulation used single pulses or pulse trains [30, 40-51]. In the one exception, Baumbauer et al. activated ChR2-expressing keratinocytes with sustained light [52]. Single pulses and pulse trains are just some of the many possible waveforms, especially since LEDs can be so easily controlled. Notably, just like pulsed electrical stimuli lost favor in pain testing because of the unnaturally synchronized neural activation they evoke [1], pulsed optogenetic stimuli warrant similar scrutiny and should not be the only waveform tested. Indeed different rates of radiant heating differentially engage C- and A-fibers [64], thus enabling the role of different afferents to be studies. By testing photostimulus ramps, we uncovered genotypic differences that were not evident with pulses. The basis for the genotypic difference requires further investigation but we hypothesize that co-activation of non-nociceptive afferents in Advillin- and TRPV1-ChR2 mice (but not in Nav1.8-ChR2 mice) engages a gate control mechanism that tempers the effects of nociceptive input, consistent with Arcourt et al. [30], who showed that activating Aδ-HTMRs in isolation evoked more guarding, jumping and vocalization than co-activating Aδ-HTMRs and LTMRS.

By testing different photostimulus waveforms, one can start to delineate the underlying interactions. Photostimulus kinetics influence how optogenetic actuators like ChR2 respond (e.g., whether they desensitize, thus producing less current for a given photostimulus intensity), but one must also consider how neurons respond to those photocurrents. Specifically, pulsed stimuli tend to evoke precisely timed spikes [40], leading to spikes that are synchronized across co-activated neurons [74], which may or may not accurately reflect the spiking patterns evoked by somatosensory stimuli. Artificial stimuli need not mimic natural stimuli to be informative; indeed, deliberately evoking spiking patterns not possible with natural stimuli offers new opportunities to probe somatosensory coding, including the role of synchrony. In that respect, optogenetic testing should not be limited to pulsed photostimuli. Interestingly, some studies [46, 47] have tested if ChR2-expressing mice avoid blue-lit floors, which they do. In these cases, the floor light was continuous, unlike the pulses typically applied by fiber optic; it is, therefore, notable that mice avoided the blue floor but did not respond to it with reflexive withdrawal, paw licking, or other outward signs of pain,

like they did to pulses. In another case where the floor light was pulsed [49], reflexive withdrawal was observed. These results highlight the underappreciated importance of the stimulus waveform.

To summarize, we describe a new device capable of reproducible, automated, multimodal algometry. Aiming, stimulation, and measurement are fully automated, which improves standardization and increases throughput, amongst other benefits. A video record of the animal before, during and after stimulation allows one to extend analysis beyond traditional response metrics (i.e. threshold and latency) to consider if evoked and ongoing pain behaviors are correlated.

### Design Considerations

Fully automated testing of withdrawal reflexes requires automation of all steps, including aiming, stimulus delivery, and response measurement. Automation of each step might be straightforward when considered in isolation, but the most obvious solutions are incompatible when combined to automate the entire process. Compatible solutions are not obvious, as explained below.

Automated aiming requires: (1) a clear view of the mouse from below, (2) real-time processing of the video using AI to identify the target paw, and (3) motorized control of stimulator position. Requirement 3 is trivial. Requirement 2 is made possible thanks to advances in AI and computing power. Requirement 1 presents several challenges.

Requirement 1 is incompatible with typically used photostimulation methods, where a scattering light source (e.g. fiber optic cable or lamp) is positioned close to the target in order to deliver sufficient light. Proximity is important because the light rays diverge from the light source. For an unobstructed view, we moved the light source away from the target and focussed the otherwise scattering light.

More notably, requirement 1 is also incompatible with obvious methods to not only detect the paw, but to detect its movement with high temporal resolution. Photoelectric sensors can work at sufficiently long distances, but through-beam and retro-reflective geometries are incompatible with mouse positioning, leaving only the diffused option, where light is reflected off the object back towards the light source. The detector senses how much light is reflected back. This amounts to a form of LIDAR (light detection and ranging) using signal strength—as opposed to triangulation, phase detection, or time of flight—to detect the target paw.

Importantly, the sensor must sensitively and specifically detect movement of the target paw. Sensitivity entails not failing to detect movements because they are too subtle (i.e. avoiding false negative responses). Specificity entails not confusing movement of other body parts with movement of the target paw (i.e., avoiding false positive responses). Specificity is achieved by targeting red light, whose reflectance is being measured, specifically to the target paw so that the reflectance signal originates almost entirely from that paw, and only that paw. This is feasible because the red light is focused onto the paw together with the photostimulation (blue and/or IR) light already being aimed there. Importantly, it is not practical to shine red light and measure its reflectance from above because the paw is typically not visible under the body. Nor is it practical to shine red light and measure its reflectance from the side (including the front or rear) because variations in mouse orientation end up requiring 360° access, or extraordinary measures must be taken to keep the mouse facing in a preferred orientation. The only efficient means of response measurement involves direct visualization and targeting of the paw from below.

The reflectance signal depends on: (i) target distance, (ii) target size, (iii) aspect, and (iv) reflectivity. During paw withdrawal, paw reflectivity does not change and changes in paw distance and aspect are minor, whereas the target “size”, namely how much of the paw remains inside the spot of red light, changes significantly (see FIG. 1C). Reflectance of red light off other body parts, which may be exposed to the light when the target paw moves, have different reflectivity and aspect, all of which combine to produce a robust change in reflectance signal that is both sensitive and specific to paw withdrawal. Paw withdrawal could be detected by video, but doing so with the high (millisecond) temporal precision needed for this application would require high-speed video. The cost and data processing required for reflectance-based latency measurements is far less than for high-speed video. Also, real-time processing of the reflectance signal enables closed loop control of stimulation such that stimuli can be automatically terminated upon detection of paw withdrawal.

Hence, by moving the light sources away from the target paw and implementing a sensor that detects paw withdrawal sensitively, specifically, and with high temporal precision at long range (i.e. the same range used for stimulation optics), the view of the paw from below is left unobstructed. This yields an unobstructed view for video from which neural networks can be trained to recognized multiple key points, including the target paw.

In the case of mechanostimulation, the mechanical probe (indenter arm) must make physical contact with target paw. This partially obscures the view of the paw from below. Moreover, because the indenter arm cannot pass through the plexiglass platform used for photostimulation, we switched to a metal grate platform when testing mechanical stimuli. The metal bars also partially obscure the view of the target paw from below. Steps are taken to minimize the obstruction, which includes using widely space bars and a narrow indenter arm, but it is still not obvious that a neural network could recognize the target paw for the purpose of automated aiming under these conditions. We have demonstrated that a neural network trained under the appropriate conditions can still perform extremely well. Moreover, we have shown that withdrawal measurement does not require a reflectance signal under these stimulus conditions because changes in the force exerted by the indenter arm on the target paw, which can be measured at high rate, indicate when withdrawal occurs and at what force.

In an embodiment the present disclosure provides an apparatus for automated measurement of pain and responses to various somatosensory stimuli in laboratory rodents, comprising:

one or more enclosures for individual rodents;

a platform on which said one or more enclosures are positioned;

a moveable device positioned underneath said platform and enclosures and configured to:
aim at a target paw of the rodent,
deliver one or more different stimulus modalities, alone or in combination, to the target paw,
detect changes in position of the target paw with millisecond precision, and
collect video of rodent activity before, during and after stimulation; and

a controller operably connected to the moveable device and configured to:
coordinate all aspects of stimulation using programmed instructions,
synchronize recorded data with stimulus timing and calculate withdrawal latency therefrom, and
automatically record all data, metadata, and calculations to electronic files.

In an embodiment the present disclosure provides a method for automated measurement of pain and responses to various somatosensory stimuli in laboratory rodents, comprising:

confining one or more rodents individually in one or more enclosures in which said one or more enclosures are located on a platform;

directing a moveable device positioned underneath said platform and enclosures to:
aim different sources of stimulation, alone or in combination, at a target paw of the rodent
deliver one or more different stimulus modalities, alone or in combination, to the target paw,
detect changes in position of the target paw with millisecond precision, and
collect video of rodent activity before, during and after stimulation; and using a controller operably connected to the moveable device to:
   coordinate all aspects of stimulation using programmed instructions,
   synchronize recorded data with stimulus timing and calculate withdrawal latency therefrom, and
   automatically record all data, metadata, and calculations to electronic files.

In an embodiment the enclosures each comprise a separate clear tube and opaque cubicle, wherein the clear tube is used to transfer each rodent from its home cage to the testing platform and to house the rodent during testing on the platform, and wherein the opaque, magnetically connectable cubicles separate the rodents and position them at a desired spacing and alignment on the platform.

In an embodiment the platform is made of an optically clear material, and wherein the moveable device may include a light source of selected wavelength(s) to provide optogenetic stimulation.

In an embodiment the platform is made of an optically clear material, and wherein the moveable device may include infrared (IR) light for thermal stimulation via radiant heating.

In an embodiment the platform is a metal grating, and the moveable device may include a mechanical indenter which stimulates by physical contact with the target paw.

In an embodiment the mechanical indenter is configured to measure force applied to the paw and to detect withdrawal based on changes in force as the target paw is withdrawn from the indenter arm.

In an embodiment the mechanical indenter is adapted to provide other somatosensory modalities requiring contact with the paw, including:
heating or cooling using a Peltier device,
application of chemicals like acetone for cooling or capsaicin for heating,
needle prick using a sharp-tipped probe, and
dynamic touch using a rotary brush.

In an embodiment the one or more stimulus modalities include combinations of light, heat, mechanical and chemical agents.

In an embodiment the moveable device is configured to provide different stimulus modalities sequentially to test different stimulus modalities on separate trials.

In an embodiment the moveable device is configured to provide two or more different stimulus modalities together on a given trial.

In an embodiment the moveable device includes a source of red light configured to be aimed at the target paw in order to assist aiming by identifying a photostimulation zone prior to initiating photostimulation with other wavelengths of light.

In an embodiment the moveable device is mounted on a set of motorized actuators and is aimed at the target paw by a human operator via computer using a joystick or keypad.

In an embodiment the moveable device is mounted on a set of motorized actuators and is aimed at the target paw automatically by a neural network pre-trained to recognize and track the target paw.

In an embodiment the initiation of stimulation made contingent on various factors ascertained from video and assessed by artificial intelligence, such as whether the rodent is stationary, has assumed a certain posture, and/or is engaged in a certain behavior.

In an embodiment the software coordinates interleaved testing of a cohort of rodents positioned on the platform so that many rodents can be rapidly tested sequentially, but where each rodent is not re-tested before a minimum acceptable period has elapsed, thus enabling high-throughput testing of the cohort.

In an embodiment a red light source is used to illuminate the target paw and a photodetector is used to measure changes in the reflectance of red light off the target paw before, during and after stimulation in order to detect withdrawal of the target paw with millisecond precision.

### CONCLUSION

The present inventors have developed a device able to reproducibly deliver photostimuli of different wavelengths, intensities, and kinetics (waveforms). This is a significant improvement over the handheld fiber optics typically used for transcutaneous optogenetic stimulation. The present inventors have also incorporated a low-cost, high-speed photometer to detect paw withdrawal and measure withdrawal latency with millisecond precision based on changes in the reflectance of red light. The accuracy of this approach was validated by comparison with high-speed video. Closed-loop control of stimulation based on automated detection of reflectance changes is made possible by real-time data processing. The inventors also incorporated computer-controlled mechanical stimulation and automated detection of touch-evoked withdrawal. Building on computer-controlled stimulation and response measurement plus video-based aiming, they automated the aiming process by training neural networks to recognize the target paw and track its location, and that information was in turn used to control motorized actuators to reposition the stimulator. Whereas aiming by joystick prevents the tester from working in close proximity to the mice, which stresses them, automating the process removes the human element altogether, with significant benefits in terms of standardization, objectivity, and throughput. Video data also allows for quantification of associated non-reflexive behaviors and their correlation with withdrawal metrics, and also allows for stimulation to be made contingent on the mouse being in a certain posture or behavioral state. Automation also facilitates standardized recording of data and metadata, which is crucial for the creation of large data sets amenable to future mining.

### REFERENCES

[1] Le Bars, D., Gozariu, M., and Cadden, S. W. (2001). Animal models of nociception. Pharmacol Rev 53, 597-657.

[2] Deuis, J. R., Dvorakova, L. S., and Vetter, I. (2017). Methods used to evaluate pain behaviors in rodents. Front Mol Neurosci 10, 284. 10.3389/fnmol.2017.00284.

[3] Barrot, M. (2012). Tests and models of nociception and pain in rodents. Neuroscience 211, 39-50. 10.1016/J. NEUROSCIENCE.2011.12.041.

[4] Gregory, N. S., Harris, A. L., Robinson, C. R., Dougherty, P. M., Fuchs, P. N., and Sluka, K. A. (2013). An overview of animal models of pain: disease models and outcome measures. J Pain 14, 1255-1269. 10.1016/J.JPAIN.2013.06.008.

[5] Burma, N. E., Leduc-Pessah, H., Fan, C. Y., and Trang, T. (2017). Animal models of chronic pain: Advances and challenges for clinical translation. J Neurosci Res 95, 1242-1256. 10.1002/JNR.23768.

[6] Jaggi, A. S., Jain, V., and Singh, N. (2011). Animal models of neuropathic pain. Fundam Clin Pharmacol 25, 1-28. 10.1111/J.1472-8206.2009.00801.X.

[7] Abboud, C., Duveau, A., Bouali-Benazzouz, R., Massé, K., Mattar, J., Brochoire, L., Fossat, P., Boué-Grabot, E., Hleihel, W., and Landry, M. (2021). Animal models of pain: Diversity and benefits. J Neurosci Methods 348, 108997. 10.1016/J.JNEUMETH.2020.108997.

[8] Mogil, J. S., and Crager, S. E. (2004). What should we be measuring in behavioral studies of chronic pain in animals? Pain 112, 12-15. 10.1016/J.PAIN.2004.09.028.

[9] Backonja, M. M., and Stacey, B. (2004). Neuropathic pain symptoms relative to overall pain rating. J Pain 5, 491-497. 10.1016/J.JPAIN.2004.09.001.

[10] Maier, C., Baron, R., Tölle, T. R., Binder, A., Birbaumer, N., Birklein, F., Gierthmühlen, J., Flor, H., Geber, C., Huge, V., et al. (2010). Quantitative sensory testing in the German Research Network on Neuropathic Pain (DFNS): Somatosensory abnormalities in 1236 patients with different neuropathic pain syndromes. Pain 150, 439-450. 10.1016/J.PAIN.2010.05.002.

[11] Koltzenburg, M., Torebjörk, H. E., and Wahren, L. K. (1994). Nociceptor modulated central sensitization causes mechanical hyperalgesia in acute chemogenic and chronic neuropathic pain. Brain 117, 579-591. 10.1093/BRAIN/117.3.579.

[12] Rowbotham, M. C., and Fields, H. L. (1996). The relationship of pain, allodynia and thermal sensation in post-herpetic neuralgia. Brain 119, 347-354. 10.1093/BRAIN/119.2.347.

[13] Pitzer, C., Kuner, R., and Tappe-Theodor, A. (2016). Voluntary and evoked behavioral correlates in neuropathic pain states under different social housing conditions. Mol Pain 12. 10.1177/1744806916656635.

[14] Mogil, J. S., Graham, A. C., Ritchie, J., Hughes, S. F., Austin, J. S., Schorscher-Petcu, A., Langford, D. J., and Bennett, G. J. (2010). Hypolocomotion, asymmetrically directed behaviors (licking, lifting, flinching, and shaking) and dynamic weight bearing (gait) changes are not measures of neuropathic pain in mice. Mol Pain 6. 10.1186/1744-8069-6-34.

[15] Edwards, R. R., Dworkin, R. H., Turk, D. C., Angst, M. S., Dionne, R., Freeman, R., Hansson, P., Haroutounian, S., Arendt-Nielsen, L., Attal, N., et al. (2016). Patient phenotyping in clinical trials of chronic pain treatments: IMMPACT recommendations. Pain 157, 1851-1871. 10.1097/J.PAIN.0000000000000602.

[16] Baron, R., Dickenson, A. H., Calvo, M., Dib-Hajj, S. D., and Bennett, D. L. (2022). Maximizing treatment efficacy through patient stratification in neuropathic pain trials. Nat Rev Neurol 19, 53-64. 10.1038/S41582-022-00741-7.

[17] Arnold, L. M., Bennett, R. M., Crofford, L. J., Dean, L. E., Clauw, D. J., Goldenberg, D. L., Fitzcharles, M. A., Paiva, E. S., Staud, R., Sarzi-Puttini, P., et al. (2019). AAPT Diagnostic Criteria for Fibromyalgia. J Pain 20, 611-628. 10.1016/J.JPAIN.2018.10.008.

[18] Negus, S. S. (2019). Core Outcome Measures in Preclinical Assessment of Candidate Analgesics. Pharmacol Rev 71, 225-266. 10.1124/PR.118.017210.

[19] Chesler, E. J., Wilson, S. G., Lariviere, W. R., Rodriguez-Zas, S. L., and Mogil, J. S. (2002). Influences of laboratory environment on behavior. Nat Neurosci 5, 1101-1102. 10.1038/NN1102-1101.

[20] Sadler, K. E., Mogil, J. S., and Stucky, C. L. (2021). Innovations and advances in modelling and measuring pain in animals. Nat Rev Neurosci 23, 70-85. 10.1038/S41583-021-00536-7.

[21] Mogil, J. S. (2017). Laboratory environmental factors and pain behavior: the relevance of unknown unknowns to reproducibility and translation. Lab Anim (NY) 46, 136-141. 10.1038/laban.1223.

[22] Chaplan, S. R., Bach, F. W., Pogrel, J. W., Chung, J. M., and Yaksh. T. L. (1994). Quantitative assessment of tactile allodynia in the rat paw. J Neurosci Methods 53, 55-63.

[23] Hargreaves, K., Dubner, R,, Brown, F., Flores, C., and Joris, J. (1988). A new and sensitive method for measuring thermal nociception in cutaneous hyperalgesia Pain 32, 77-88.

[24] Le Bars, D., Hansson, P. T., and Plaghki, L. (2009). Current animal test and models of pain. In Pharmacology of Pain, pp. 475-504.

[25] Fried, N. T., Chamessian, A., Zylka, M. J., and Abdus-Saboor, I. (2020). Improving pain assessment in mice and rats with advanced videography and computational approaches. Pain 161. 10.1097/j.pain.0000000000001843.

[26] Jones, J. M., Foster, W., Twomey, C. R., Burdge, J., Ahmed, O. M., Pereira, T. D., Wojick, J. A., Corder, G., Plotkin, J. B., and Abdus-Saboor, I. (2020). A machine-vision approach for automated pain measurement at millisecond timescales. Elife 9. 10.7554/eLife.57258.

[27] Abdus-Saboor, I., Fried, N. T., Lay, M., Burdge, J., Swanson, K., Fischer, R., Jones, J., Dong, P., Cai, W., Guo, X., et al. (2019). Development of a Mouse Pain Scale Using Sub-second Behavioral Mapping and Statistical Modeling. Cell Rep 28, 1623-1634.e4. 10.1016/j.celrep.2019.07.017.

[28] Copits, B. A., Pullen, M. Y., and Gereau, R .W. (2016). Spotlight on pain: Optogenetic approaches for interrogating somatosensory circuits. Pain 157, 2424-2433. 10.1097/J.PAIN.0000000000000620.

[29] Xie, Y. F., Wang, J., and Bonin, R. P. (2018). Optogenetic exploration and modulation of pain processing. Exp Neurol 306, 117-121. 10.1016/J.EXPNEUROL.2018.05.003.

[30] Arcourt, A., Gorham, L., Dhandapani, R., Prato, V., Taberner, F. J., Wende, H., Gangadharan, V., Birchmeier, C., Heppenstall, P. A., and Lechner, S. G. (2017). Touch Receptor-Derived Sensory Information Alleviates Acute Pain Signaling and Fine-Tunes Nociceptive Reflex Coordination. Neuron 93, 179-193. 10.1016/j.neuron.2016.11.027.

[31] Prescott, S. A., and Ratté, S. (2012). Pain processing by spinal microcircuits: afferent combinatorics. Curr Opin Neurobiol, 631-639. 10.1016/j.conb.2012.02.010.

[32] Woolf, C. J. (2020). Capturing Novel Non-opioid Pain Targets. Biol Psychiatry 87, 74-81. 10.1016/J.BIOPSYCH.2019.06.017.

[33] Hurst, J. L., and West, R. S. (2010). Taming anxiety in laboratory mice. Nat Methods 7, 825-826. 10.1038/nmeth. 1500.

[34] Gouveia, K., and Hurst, J. L. (2013). Reducing Mouse Anxiety during Handling: Effect of Experience with Handling Tunnels. PLOS One 8, 66401. 10.1371/JOURNAL.PONE.0066401.

[35] Gouveia, K., and Hurst, J. L. (2019). Improving the practicality of using non-aversive handling methods to reduce background stress and anxiety in laboratory mice. Scientific Reports 2019 9: 1 9, 1-19. 10.1038/s41598-019-56860-7.

[36] De Farias Rocha, F. A., Gomes, B. D., De Lima Silveira, L. C., Martins, S. L., Aguiar, R. G., De Souza, J. M., and Ventura, D. F. (2016). Spectral Sensitivity Measured with Electroretinogram Using a Constant Response Method. PLOS One 11, e0147318. 10.1371/JOURNAL.PONE.0147318.

[37] Nikbakht, N., and Diamond, M. E. (2021). Conserved visual capacity of rats under red light. Elife 10. 10.7554/ELIFE.66429.

[38] Niklaus, S., Albertini, S., Schnitzer, T. K., and Denk, N. (2020). Challenging a Myth and Misconception: Red-Light Vision in Rats. Animals 10, 422. 10.3390/ANI10030422.

[39] Mathis, A., Mamidanna, P., Cury, K. M., Abe, T., Murthy, V. N., Mathis, M. W., and Bethge, M. (2018). DeepLabCut: markerless pose estimation of user-defined body parts with deep learning. Nature Neuroscience 2018 21:9 21, 1281-1289. 10.1038/s41593-018-0209-y.

[40] Browne, L. E., Latremoliere, A., Lehnert, B. P., Grantham, A., Ward, C., Alexandre, C., Costigan, M., Michoud, F., Roberson, D. P., Ginty, D. D., et al. (2017). Time-Resolved Fast Mammalian Behavior Reveals the Complexity of Protective Pain Responses. Cell Rep 20, 89-98. 10.1016/j.celrep.2017.06.024.

[41] Dhandapani, R., Arokiaraj, C. M., Taberner, F. J., Pacifico, P., Raja, S., Nocchi, L., Portulano, C., Franciosa, F., Maffei, M., Hussain, A. F., et al. (2018). Control of mechanical pain hypersensitivity in mice through ligand-targeted photoablation of TrkB-positive sensory neurons. Nat Commun 9, 1-14. 10.1038/s41467-018-04049-3.

[42] Chamessian, A., Matsuda, M., Young, M., Wang, M., Zhang, Z. J., Liu, D., Tobin, B., Xu, Z. Z., Van de Ven, T., and Ji, R. R. (2019). Is Optogenetic Activation of Vglut1-Positive AB Low-Threshold Mechanoreceptors Sufficient to Induce Tactile Allodynia in Mice after Nerve Injury? The Journal of neuroscience 39, 6202-6215. 10.1523/JNEUROSCI.2064-18.2019.

[43] Warwick, C., Cassidy, C., Hachisuka, J., Wright, M. C., Baumbauer, K. M., Adelman, P. C., Lee, K. H., Smith, K. M., Sheahan, T. D., Ross, S. E., et al. (2021). Mrgprd Cre lineage neurons mediate optogenetic allodynia through an emergent polysynaptic circuit. Pain 162, 2120-2131. 10.1097/j.pain.0000000000002227.

[44] Beaudry, H., Daou, I., Ase, A. R., Ribeiro-Da-Silva, A., and Séguela, P. (2017). Distinct behavioral responses evoked by selective optogenetic stimulation of the major TRPV1+ and MrgD+ subsets of C-fibers. Pain 158, 2329-2339. 10.1097/J.PAIN.0000000000001016.

[45] Schorscher-Petcu, A., Takács, F., and Browne, L. E. (2021). Scanned optogenetic control of mammalian somatosensory input to map input-specific behavioral outputs. Elife 10. 10.7554/ELIFE.62026.

[46] Daou, I., Tuttle, A. H., Longo, G., Wieskopf, J. S., Bonin, R. P., Ase, A. R., Wood, J. N., De Koninck, Y., Ribeiro-da-Silva, A., Mogil, J. S., et al. (2013). Remote Optogenetic Activation and Sensitization of Pain Pathways in Freely Moving Mice. The Journal of Neuroscience 33, 18631. 10.1523/JNEUROSCI.2424-13.2013.

[47] Iyer, S. M., Montgomery, K. L., Towne, C., Lee, S. Y., Ramakrishnan, C., Deisseroth, K., and Delp, S. L. (2014). Virally mediated optogenetic excitation and inhibition of pain in freely moving nontransgenic mice. Nature Biotechnology 2014 32: 3 32, 274-278. 10.1038/NBT.2834.

[48] Abdo, H., Calvo-Enrique, L., Lopez, J. M., Song, J., Zhang, M. D., Usoskin, D., Manira, A. El, Adameyko, I., Hjerling-Leffler, J., and Ernfors, P. (2019). Specialized cutaneous schwann cells initiate pain sensation. Science (1979) 365, 695-699. 10.1126/SCIENCE.AAX6452.

[49] Barik, A., Thompson, J. H., Seltzer, M., Ghitani, N., and Chesler, A. T. (2018). A Brainstem-Spinal Circuit Controlling Nocifensive Behavior. Neuron 100, 1491-1503.e3. 10.1016/J.NEURON.2018.10.037.

[50] Sharif, B., Ase, A. R., Ribeiro-da-Silva, A., and Séguéla, P. (2020). Differential Coding of Itch and Pain by a Subpopulation of Primary Afferent Neurons. Neuron 106, 940-951.e4. 10.1016/J.NEURON.2020.03.021.

[51] Tashima, R., Koga, K., Sekine, M., Kanehisa, K., Kohro, Y., Tominaga, K., Matsushita, K., Tozaki-Saitoh, H., Fukazawa, Y., Inoue, K., et al. (2018). Optogenetic Activation of Non-Nociceptive AB Fibers Induces Neuropathic Pain-Like Sensory and Emotional Behaviors after Nerve Injury in Rats. eNeuro 5. 10.1523/ENEURO.0450-17.2018.

[52] Baumbauer, K. M., Deberry, J. J., Adelman, P. C., Miller, R. H., Hachisuka, J., Lee, K. H., Ross, S. E., Koerber, H. R., Davis, B. M., and Albers, K. M. (2015). Keratinocytes can modulate and directly initiate nociceptive responses. Elife 4. 10.7554/ELIFE.09674.

[53] Zhou, X., Wang, L., Hasegawa, H., Amin, P., Han, B. X., Kaneko, S., He, Y., and Wang, F. (2010). Deletion of PIK3C3/Vps34 in sensory neurons causes rapid neurodegeneration by disrupting the endosomal but not the autophagic pathway. Proc Natl Acad Sci U S A 107, 9424-9429. 10.1073/PNAS.0914725107.

[54] Nassar, M. A., Levato, A., Stirling, L. C., and Wood, J. N. (2005). Neuropathic pain develops normally in mice lacking both Nav 1.7 and Nav 1.8. Mol Pain 1. 10.1186/1744-8069-1-24.

[55] Agarwal, N., Offermanns, S., and Kuner, R. (2004). Conditional gene deletion in primary nociceptive neurons of trigeminal ganglia and dorsal root ganglia. genesis 38, 122-129. 10.1002/GENE.20010.

[56] Hires, A. S., Gutnisky, D. A., Yu, J., O'Connor, D. H., and Svoboda, K. (2015). Low-noise encoding of active touch by layer 4 in the somatosensory cortex. Elife 4, e06619. 10.7554/eLife.06619.

[57] Kane, G. A., Lopes, G., Saunders, J. L., Mathis, A., and Mathis, M. W. (2020). Real-time, low-latency closed-loop feedback using markerless posture tracking. Elife 9, 1-29. 10.7554/ELIFE.61909.

[58] Kauppila, T., Kontinen, V. K., and Pertovaara, A. (1998). Weight bearing of the limb as a confounding factor in assessment of mechanical allodynia in the rat. Pain 74, 55-59. 10.1016/S0304-3959(97)00143-7.

[59] Sorge, R. E., Martin, L. J., Isbester, K. A., Sotocinal, S. G., Rosen, S., Tuttle, A. H., Wieskopf, J. S., Acland, E. L., Dokova, A., Kadoura, B., et al. (2014). Olfactory exposure to males, including men, causes stress and related analgesia in rodents. Nat Methods 11, 629-632. 10.1038/nmeth.2935.

[60] Usoskin, D., Furlan, A., Islam, S., Abdo, H., Lönnerberg, P., Lou, D., Hjerling-Leffler, J., Haeggström, J., Kharchenko, O., Kharchenko, P. V, et al. (2015). Unbiased classification of sensory neuron types by large-scale single-cell RNA sequencing. Nature Publishing Group 18, 145-153. 10.1038/nn.3881.

[61] Prescott, S. A., Ma, Q., and De Koninck, Y. (2014). Normal and abnormal coding of somatosensory stimuli causing pain. Nat Neurosci 17, 183-191. 10.1038/nn.3629.

[62] Mickle, A. D., Won, S. M., Noh, K. N., Yoon, J., Meacham, K. W., Xue, Y., Mcllvried, L. A., Copits, B. A., Samineni, V. K., Crawford, K. E., et al. (2019). A wireless closed-loop system for optogenetic peripheral neuromodulation. Nature 565, 361-365. 10.1038/S41586-018-0823-6.

[63] Iyer, S. M., Vesuna, S., Ramakrishnan, C., Huynh, K., Young, S., Berndt, A., Lee, S. Y., Gorini, C. J., Deisseroth, K., and Delp, S. L. (2016). Optogenetic and chemogenetic strategies for sustained inhibition of pain. Scientific Reports 2016 6:1 6, 1-10. 10.1038/SREP30570.

[64] Plaghki, L., Decruynaere, C., Van Dooren, P., and Le Bars, D. (2010). The Fine Tuning of Pain Thresholds: A Sophisticated Double Alarm System. PLOS One 5, 10269. 10.1371/JOURNAL.PONE.0010269.

[65] Hsu, A. I., and Yttri, E. A. (2021). B-SOiD, an open-source unsupervised algorithm for identification and fast prediction of behaviors. Nature Communications 2021 12:1 12, 1-13. 10.1038/S41467-021-25420-X.

[66] Luxem, K., Mocellin, P., Fuhrmann, F., Kürsch, J., Miller, S. R., Palop, J. J., Remy, S., and Bauer, P. (2022). Identifying behavioral structure from deep variational embeddings of animal motion. Communications Biology 2022 5:1 5, 1-15. 10.1038/S42003-022-04080-7.

[67] Pereira, T. D., Tabris, N., Matsliah, A., Turner, D. M., Li, J., Ravindranath, S., Papadoyannis, E. S., Normand, E., Deutsch, D. S., Wang, Z. Y., et al. (2022). SLEAP: A deep learning system for multi-animal pose tracking. Nature Methods 2022 19:4 19, 486-495. 10.1038/S41592-022-01426-1.

[68] Graving, J. M., Chae, D., Naik, H., Li, L., Koger, B., Costelloe, B. R., and Couzin, I. D. (2019). Deepposekit, a software toolkit for fast and robust animal pose estimation using deep learning. Elife 8. 10.7554/ELIFE.47994.

[69] Weinreb, C., Abdal, M., Osman, M., Zhang, L., Lin, S., Pearl, J., Annapragada, S., Conlin, E., Gillis, W. F., Jay, M., et al. (2023). Keypoint-MoSeq: parsing behavior by linking point tracking to pose dynamics. bioRxiv, 2023.03.16.532307. 10.1101/2023.03.16.532307.

[70] Bohnslav, J. P., Wimalasena, N. K., Clausing, K. J., Dai, Y. Y., Yarmolinsky, D. A., Cruz, T., Kashlan, A. D., Chiappe, M. E., Orefice, L. L., Woolf, C. J., et al. (2021). DeepEthogram, a machine learning pipeline for supervised behavior classification from raw pixels. Elife 10. 10.7554/ELIFE.63377.

[71] Bohic, M., Pattison, L. A., Jhumka, Z. A., Rossi, H., Thackray, J. K., Ricci, M., Mossazghi, N., Foster, W., Ogundare, S., Twomey, C. R., et al. (2023). Mapping the neuroethological signatures of pain, analgesia, and recovery in mice. Neuron. https://doi.org/10.1016/j.neuron.2023.06.008.

[72] Zhang, Z., Roberson, D. P., Kotoda, M., Boivin, B., Bohnslav, J. P., González-Cano, R., Yarmolinsky, D. A., Turnes, B. L., Wimalasena, N. K., Neufeld, S. Q., et al. (2022). Automated preclinical detection of mechanical pain hypersensitivity and analgesia. Pain 163, 2326-2336. 10.1097/J.PAIN.0000000000002680.

[73] Blivis, D., Haspel, G., Mannes, P. Z ., O'Donovan, M. J., and Iadarola, M. J. (2017). Identification of a novel spinal nociceptive-motor gate control for Ao pain stimuli in rats. Elife 6. 10.7554/ELIFE.23584.

[74] Ratté, S., Hong, S., DeSchutter, E., and Prescott, S. A. (2013). Impact of neuronal properties on network coding: Roles of spike initiation dynamics and robust synchrony transfer. Neuron 78, 758-772. 10.1016/j.neuron.2013.05.030

Therefore what is claimed is:

1. An apparatus for automated measurement of pain and responses to various somatosensory stimuli in laboratory rodents, comprising:
one or more enclosures for individual rodents;
a platform on which said one or more enclosures are positioned;
a moveable device positioned underneath said platform and enclosures and configured to:
aim at a target paw of the rodent,
deliver one or more different stimulus modalities, alone or in combination, to the target paw,
detect changes in position of the target paw with millisecond precision, and
collect video of rodent activity before, during and after stimulation; and
a controller operably connected to the moveable device and configured to:
coordinate all aspects of stimulation using programmed instructions,
synchronize recorded data with stimulus timing and calculate withdrawal latency therefrom, and
automatically record all data, metadata, and calculations to electronic files.

2. The apparatus according to claim 1, wherein said enclosures each comprise a separate clear tube and opaque cubicle, wherein the clear tube is used to transfer each rodent from its home cage to the testing platform and to house the rodent during testing on the platform, and wherein the opaque, magnetically connectable cubicles separate the rodents and position them at a desired spacing and alignment on the platform.

3. The apparatus according to claim 1, wherein the platform is made of an optically clear material, and wherein the moveable device includes a light source of selected wavelength(s) to provide optogenetic stimulation.

4. The apparatus according to claim 1, wherein the platform is made of an optically clear material, and wherein the moveable device includes infrared (IR) light for thermal stimulation via radiant heating.

5. The apparatus according to claim 1, wherein the platform is metal grating, and wherein the moveable device includes a mechanical indenter which stimulates by physical contact with the target paw.

6. The apparatus according to claim 5, wherein the mechanical indenter is configured to measure force applied to the paw and to detect withdrawal based on changes in force as the target paw is withdrawn from the indenter arm.

7. The apparatus according to claim 5, wherein the mechanical indenter is adapted to provide other somatosensory modalities requiring contact with the paw, including:
heating or cooling using a Peltier device,
application of chemicals including for cooling or for heating,
needle prick using a sharp-tipped probe, and
dynamic touch using a rotary brush.

8. The apparatus according to claim 1, wherein said one or more stimulus modalities include combinations of light, heat, mechanical and chemical agents.

9. The apparatus according to claim 1, wherein said moveable device is configured to provide different stimulus modalities sequentially to test different stimulus modalities on separate trials.

10. The apparatus according to claim 1, wherein said moveable device is configured to provide two or more different stimulus modalities together on a given trial.

11. The apparatus according to claim 1, wherein said moveable device includes a source of red light configured to be aimed at said target paw in order to assist aiming by identifying a photostimulation zone prior to initiating photostimulation with other wavelengths of light.

12. The apparatus according to claim 1, wherein the moveable device is mounted on a set of motorized actuators and is aimed at the target paw by a human operator via computer using a joystick or keypad.

13. The apparatus according to claim 1, wherein the moveable device is mounted on a set of motorized actuators and is aimed at the target paw automatically by a neural network pre-trained to recognize and track the target paw.

14. The apparatus according to claim 13, wherein initiation of stimulation is made contingent on various factors ascertained from video and assessed by artificial intelligence, such as whether the rodent is stationary, has assumed a certain posture, and/or is engaged in a certain behavior.

15. The apparatus according to claim 13, wherein software coordinates interleaved testing of a cohort of rodents positioned on the platform so that many rodents can be rapidly tested sequentially, but where each rodent is not re-tested before a minimum acceptable period has elapsed, thus enabling high-throughput testing of the cohort.

16. The apparatus according to claim 1, wherein a red light source is used to illuminate the target paw and a photodetector is used to measure changes in the reflectance of red light off the target paw before, during and after stimulation in order to detect withdrawal of the target paw with millisecond precision.

17. A method for automated measurement of pain and responses to various somatosensory stimuli in laboratory rodents, comprising:
confining one or more rodents individually in one or more enclosures in which said one or more enclosures are located on a platform;
directing a moveable device positioned underneath said platform and enclosures to:
aim different sources of stimulation, alone or in combination, at a target paw of the rodent
deliver one or more different stimulus modalities, alone or in combination, to the target paw,
detect changes in position of the target paw with millisecond precision, and
collect video of rodent activity before, during and after stimulation; and
using a controller operably connected to the moveable device to:
coordinate all aspects of stimulation using programmed instructions, synchronize recorded data with stimulus timing and calculate withdrawal latency therefrom, and
automatically record all data, metadata, and calculations to electronic files.

18. The method according to claim 17, wherein said enclosures each comprise a separate clear tube and opaque cubicle, wherein the clear tube is used to transfer each rodent from its home cage to the testing platform and to house the rodent during testing on the platform, and wherein the opaque, magnetically connectable cubicles separate the rodents and position them at a desired spacing and alignment on the platform.

19. The method according to claim 17, wherein the platform is made of an optically clear material, and wherein the moveable device includes a light source of selected wavelength(s) to provide optogenetic stimulation.

20. The method according to claim 17, wherein the platform is made of an optically clear material, and wherein the moveable device includes infrared (IR) light for thermal stimulation via radiant heating.

21. The method according to claim 17, wherein the platform is metal grating, and wherein the moveable device includes a mechanical indenter which stimulates by physical contact with the target paw.

22. The method according to claim 21, wherein the mechanical indenter is configured to measure force applied to the paw and to detect withdrawal based on changes in force as the target paw is withdrawn from the indenter arm.

23. The method according to claim 21, wherein the mechanical indenter is adapted to provide other somatosensory modalities requiring contact with the paw, including:
heating or cooling using a Peltier device,
application of chemicals like acetone for cooling or capsaicin for heating
needle prick using a sharp-tipped probe, and
dynamic touch using a rotary brush.

24. The method according to claim 17, wherein said moveable device includes a source of red light configured to be aimed at said target paw in order to assist aiming by identifying a photostimulation zone prior to initiating photostimulation with other wavelengths of light.

25. The method according to claim 17, wherein the moveable device is mounted on a set of motorized actuators and is aimed at the target paw by a human operator via computer using a joystick or keypad.

26. The method according to claim 17, wherein the moveable device is mounted on a set of actuators and the stimulus is aimed at the target paw automatically by a neural network pre-trained to recognize and track the target paw.

27. The method according to claim 28, wherein initiation of stimulation is made contingent on various factors ascertained from video and assessed by artificial intelligence, such as whether the rodent is stationary, has assumed a certain posture, and/or is engaged in a certain behavior.

28. The method according to claim 28, wherein software coordinates interleaved testing of a cohort of rodents positioned on the platform so that many rodents can be rapidly tested sequentially, but where each rodent is not re-tested before a minimum acceptable period has elapsed, thus enabling high-throughput testing of the cohort.

29. The method according to claim 17, wherein a red light source is used to illuminate the target paw and a photodetector is used to measure changes in the reflectance of red light off the target paw before, during and after stimulation in order to detect withdrawal of the target paw with millisecond precision.
